SEQUENCE LISTING (34 sequences)

SEQ ID NO: 1 - DNA, 1643 bp, HUMAN
gcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaattgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgctt

SEQ ID NO: 2 - DNA, 1601 bp, HUMAN
aaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 3 - DNA, 2335 bp, HUMAN
gaattccccccccccccccccgagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagggaattc

SEQ ID NO: 4 - DNA, 816 bp, HUMAN
atgcactttctttgccaaaggcaaacgcagaacgtttcagagccatgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagttgagactaaactggtttgttgcagccaaagattttggaggagaaggacattttactgcagtgagaatgagggccaagaaagagtcaggccttaattttcaatataatttaacttcagagggaaagtaaatatttcaggcatactgacactttgccagaaagcataaaattcttaaaatatatttcagatatcagaatcattgaagtattttcctccaggcaaaattgatatacttttttcttatttaacttaacattctgtaaaatgtctgttaacttaatagtatttatgaaatggttaagaatttggtaaattagtatttatttaatgttatgttgtgttctaataaaacaaaaatagacaactgttc

SEQ ID NO: 5 - DNA, 825 bp, HUMAN
atcactctctttaatcactactcacattaacctcaactcctgccacaatgtacaggatgcaactcctgtcttgcattgcactaattcttgcacttgtcacaaacagtgcacctacttcaagttcgacaaagaaaacaaagaaaacacagctacaactggagcatttactgctggatttacagatgattttgaatggaattaataattacaagaatcccaaactcaccaggatgctcacatttaagttttacatgcccaagaaggccacagaactgaaacagcttcagtgtctagaagaagaactcaaacctctggaggaagtgctgaatttagctcaaagcaaaaactttcacttaagacccagggacttaatcagcaatatcaacgtaatagttctggaactaaagggatctgaaacaacattcatgtgtgaatatgcagatgagacagcaaccattgtagaatttctgaacagatggattaccttttgtcaaagcatcatctcaacactaacttgataattaagtgcttcccacttaaaacatatcaggccttctatttatttatttaaatatttaaattttatatttattgttgaatgtatggttgctacctattgtaactattattcttaatcttaaaactataaatatggatcttttatgattctttttgtaagccctaggggctctaaaatggtttaccttatttatcccaaaaatatttattattatgttgaatgttaaatatagtatctatgtagattggttagtaaaactatttaataaatttgataaatataaaaaaaaaaaacaaaaaaaaaaa

SEQ ID NO: 6 - DNA, 614 bp, HUMAN
gatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatct

SEQ ID NO: 7 - DNA, 2534 bp, HUMAN
gacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcataccctcaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttg

SEQ ID NO: 8 - DNA, 1059 bp, HUMAN
atggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaa

SEQ ID NO: 9 - DNA, 2745 bp, HUMAN
acctccctccgcggagcagccagacagcgagggccccggccgggggcaggggggacgccccgtccggggcaccccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggcccgccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacaccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatggcagggactctgataacacccattttaaaggttgaggaaacaagcccagagaggttaagggaggagttcctgcccaccaggaacctgctttagtgggggatagtgaagaagacaataaaagatagtagttcaggccaggcggggtgctcacgcctgtaatcctagcacttttgggaggcagagatgggaggatacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcag

SEQ ID NO: 10 - DNA, 2334 bp, HUMAN
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttgcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttt

SEQ ID NO: 11 - DNA, 1128 bp, HUMAN
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtccagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatattttatatttttaagaagtaccacttgaaacattttatgtattagttttgaaataataatggaaagtggctatgcagtttgaatatcctttgtttcagagccagatcatttcttggaaagtgtaggcttacctcaaataaatggctaactttatacatatttttaaagaaatatttatattgtatttatataatgtataaatggtttttataccaataaatggcattttaaaaaattc

SEQ ID NO: 12 - DNA, 1650 bp, HUMAN
actttcctgccccttccccggccaagcccaactccggatctcgctctccaccggatctcacccgccacacccggacaggcggctggaggaggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcacctcccagcccatccatgatagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtgagggggtactgatggagggaggggtaaaggtaagagaagctgtggaggaaaaaaatctgggggaggccgggcgtggcttgcacgcctgtaatccagcctttgggaggccaaggcaggcagttacctgagatcaggagttcaagaccagcttggccaacagcgtgaaacctcgtctctactaaaaatacaaacattagctgggcatggtggcaggcgcctgtaatcccagctactcgggaggctgaggcaggagaatcgcttgaaccctgggagacaagaggttgcagtaagctgagatcacaccactgcactccaggctgggcaataagagcgaaactccgtctcaaaaaaaaaaaaaaaaaaaaaa

SEQ ID NO: 13 - DNA, 1670 bp, HUMAN
ccaaccacaagcaccaaagcagaggggcaggcagcacaccacccagcagccagagcaccagcccagccatggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcaggaccaaaaaaaaaaaaaaaaaaaaaaaaaaaaaaaaaaaaaaaaaaaaaaa

SEQ ID NO: 14 - DNA, 2986 bp, HUMAN
gcgccccagtcgacgctgagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcgaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggcccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtccatgtcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttcacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactattgcagctccagtttcctgcagtgatcagggtcctgcaagcagtggggaagggggccaaggtattggaggactccctcccagctttggaagggtcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttttcagagacggggtctcgcaacattgcccagacttcctttgtgttagttaataaagctttctcaactgcc

SEQ ID NO: 15 - DNA, 2651 bp, HUMAN
ggaattctgtggccatactgcgaggagatcggttccgggtcggaggctacaggaagactcccactccctgaaatctggagtgaagaacgccgccatccagccaccattccaaggaggtgcaggagaacagctctgtgataccatttaacttgttgacattacttttatttgaaggaacgtatattagagcttactttgcaaagaaggaagatggttgtttccgaagtggacatcgcaaaagctgatccagctgctgcatcccaccctctattactgaatggagatgctactgtggcccagaaaaatccaggctcggtggccgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcacccgtgagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacattgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaaattaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccaatccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgttcctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctctctctctgtaataaactcatttctagcag

SEQ ID NO: 16 - DNA, 6059 bp, HUMAN
cttcagatagattatatctggagtgaaggatcctgccacctacgtatctggcatagtgtgagtcctcataaatgcttactggtttgaagggcaacaaaatagtgaacagagtgaaaatccccactaagatcctgggtccagaaaaagatgggaaacctgtttagctcacccgtgagcccatagttaaaactctttagacaacaggttgtttccgtttacagagaacaataatattgggtggtgagcatctgtgtgggggttggggtgggataggggatacggggagagtggagaaaaaggggacacagggttaatgtgaagtccaggatccccctctacatttaaagttggtttaagttggctttaattaatagcaactcttaagataatcagaattttcttaaccttttagccttactgttgaaaagccctgtgatcttgtacaaatcatttgcttcttggatagtaatttcttttactaaaatgtgggcttttgactagatgaatgtaaatgttcttctagctctgatatcctttattctttatattttctaacagattctgtgtagtgggatgagcagagaacaaaaacaaaataatccagtgagaaaagcccgtaaataaaccttcagaccagagatctattctccagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccggtaagtaacctctcagctgcttggcctgttagttagcttctgagatgagtaaaagactttacaggaaacccatagaagacatttggcaaacaccaagtgctcatacaattatcttaaaatataatctttaagataaggaaagggtcacagtttggaatgagtttcagacggttataacatcaaagatacaaaacatgattgtgagtgaaagactttaaagggagcaatagtattttaataactaacaatccttacctctcaaaagaaagatttgcagagagatgagtcttagctgaaatcttgaaatcttatcttctgctaaggagaactaaaccctctccagtgagatgccttctgaatatgtgcccacaagaagttgtgtctaagtctggttctcttttttctttttcctccagacaagagggaagcctaaaaatggtcaaaattaatattaaattacaaacgccaaataaaattttcctctaatatatcagtttcatggcacagttagtatataattctttatggttcaaaattaaaaatgagcttttctaggggcttctctcagctgcctagtctaaggtgcagggagtttgagactcacagggtttaataagagaaaattctcagctagagcagctgaacttaaatagactaggcaagacagctggttataagactaaactacccagaatgcatgacattcatctgtggtggcagacgaaacattttttattatattatttcttgggtatgtatgacaactcttaattgtggcaactcagaaactacaaacacaaacttcacagaaaatgtgaggattttacaattggctgttgtcatctatgaccttctctgggacttgggcacccggccatttcactctgactacatcatgtcaccaaacatctgatggtcttgccttttaattctcttttcgaggactgagagggagggtagcatggtagttaagagtgcaggcttcccgcattcaaaatcggttgcttactagctgtgtggctttgagcaagttactcaccctctctgtgcttcaaggtccttgtctgcaaaatgtgaaaaatatttcctgcctcataaggttgccctaaggattaaatgaatgaatgggtatgatgcttagaacagtgattggcatccagtatgtgccctcgaggcctcttaattattactggcttgctcatagtgcatgttctttgtgggctaactctagcgtcaataaaaatgttaagactgagttgcagccgggcatggtggctcatgcctgtaatcccagcattctaggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttggtttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatataggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctttctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttcta

SEQ ID NO: 17 - DNA, 1160 bp, HUMAN
cctccgacagcctctccacaggtaccatgaaggtctccgcggcacgcctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctgaccacgacgagcagcagttacaaaaaccttccccaggctggacgtggtggctcagccttgtaatcccagcactttgggaggccaaggtgggtggatcacttgaggtcaggagttcgagacagcctggccaacatgatgaaaccccatgtgtactaaaaatacaaaaaattagccgggcgtggtagcgggcgcctgtagtcccagctactcgggaggctgaggcaggagaatggcgtgaacccgggagcggagcttgcagtgagccgagatcgcgccactgcactccagcctgggcgacagagcgagactccgtctcaaaaaaaaaaaaaaaaaaaaaaaaaatacaaaaattagccgcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgcaagggctctgtgacaaggaaggaagtcagcatgcctctagaggcaaggaagggaggaacactgcactcttaagcttccgccgtctcaacccctcacaggagcttactggcaaacatgaaaaatcgggg

SEQ ID NO: 18 - DNA, 1193 bp, HUMAN
tgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcaaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaact

SEQ ID NO: 19 - DNA, 4040 bp, HUMAN
tgcagagaacagagaaaggacatctgcgaggaaagttccctgatggctgtcaacaaagtgccacgtctctatggctgtgtacgctgagcacacgattttatcgcgcctatcatatcttggtgcataaacgcacctcacctcggtcaacccttgctccgtcttatgagacaggctttattatccgcattttatatgaggggaatctgacggtggagagagaattatcttgctcaaggcgacacagcagagcccacaggtggcagaatcccacccgagcccgcttcgacccgcggggtggaaaccacgggcgcccgcccggctgcgcttccagagctgaactgagaagcgagtcctctccgccctgcggccaccgcccagccccgacccccgccccggcccgatcctcactcgccgccagctccccgcgcccaccccggagttggtggcgcagaggcgggaggcggaggcgggagggcgggcgctggcaccgggaacgcccgagcgccggcagagagcgcggagagcgcgacacgtgcggcccagagcaccggggccacccggtccccgcaggcccgggaccgcgcccgctggcaggcgacacgtggaagaatacggagttctataccagagttgattgttgatggcacatacttttagaggatgctcattggcatttatgtttataatcacgtggctgttgattaaagcaaaaatagatgcgtgcaagagaggcgatgtgactgtgaagccttcccatgtaattttacttggatccactgtcaatattacatgctctttgaagcccagacaaggctgctttcactattccagacgtaacaagttaatcctgtacaagtttgacagaagaatcaattttcaccatggccactccctcaattctcaagtcacaggtcttccccttggtacaaccttgtttgtctgcaaactggcctgtatcaatagtgatgaaattcaaatatgtggagcagagatcttcgttggtgttgctccagaacagcctcaaaatttatcctgcatacagaagggagaacaggggactgtggcctgcacctgggaaagaggacgagacacccacttatacactgagtatactctacagctaagtggaccaaaaaatttaacctggcagaagcaatgtaaagacatttattgtgactatttggactttggaatcaacctcacccctgaatcacctgaatccaatttcacagccaaggttactgctgtcaatagtcttggaagctcctcttcacttccatccacattcacattcttggacatagtgaggcctcttcctccgtgggacattagaatcaaatttcaaaaggcttccgtgagcagatgtaccctttattggagagatgagggactggtactgcttaatcgactcagatatcggcccagtaacagcaggctctggaatatggttaatgttacaaaggccaaaggaagacatgatttgctggatctgaaaccatttacagaatatgaatttcagatttcctctaagctacatctttataagggaagttggagtgattggagtgaatcattgagagcacaaacaccagaagaagagcctactgggatgttagatgtctggtacatgaaacggcacattgactacagtagacaacagatttctcttttctggaagaatctgagtgtctcagaggcaagaggaaaaattctccactatcaggtgaccttgcaggagctgacaggagggaaagccatgacacagaacatcacaggacacacctcctggaccacagtcattcctagaaccggaaattgggctgtggctgtgtctgcagcaaattcaaaaggcagttctctgcccactcgtattaacataatgaacctgtgtgaggcagggttgctggctcctcgccaggtctctgcaaactcagagggcatggacaacattctggtgacttggcagcctcccaggaaagatccctctgctgttcaggagtacgtggtggaatggagagagctccatccagggggtgacacacaggtccctctaaactggctacggagtcgaccctacaatgtgtctgctctgatttcagagaacataaaatcctacatctgttatgaaatccgtgtgtatgcactctcaggggatcaaggaggatgcagctccatcctgggtaactctaagcacaaagcaccactgagtggcccccacattaatgccatcacagaggaaaaggggagcattttaatttcatggaacagcattccagtccaggagcaaatgggctgcctcctccattataggatatactggaaggaacgggactccaactcccagcctcagctctgtgaaattccctacagagtctcccaaaattcacatccaataaacagcctgcagccccgagtgacatatgtcctgtggatgacagctctgacagctgctggtgaaagttcccacggaaatgagagggaattttgtctgcaaggtaaagccaattggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagccagaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctgagtggtgaggcttcaagccttaaagtcagtgtgccctcaaccagcacagcctgccccaattcccccagcccctgctccagcagctgtcatctctgggtgccaccatcggtctggctgcagctagaggacaggcaagccagctctgggggagtcttaggaactgggagttggtcttcactcagatgcctcatcttgcctttcccagggccttaaaattacatccttcactgtgtggacctagagactccaacttgaattcctagtaactttcttggtatgctggccagaaagggaaatgaggaggagagtagaaaccacagctcttagtagtaatggcatacagtctagaggaccattcatgcaatgactatttctaaagcacctgctacacagcaggctgtacacagcagatcagtactgttcaacagaacttcctgagatgatggaaatgttctacctctgcactcactgtccagtacattagacactaggcacattggctgttaatcacttggaatgtgtttagcttgactgaggaattaaattttgattgtaaatttaaatcgccacacatggctagtggctactgtattggagtgcacagctctagatggctcctagattattgagagcctccaaaacaaatcaacctagttctatagatgaagacataaaagacactggtaaacaccaatgtaaaagggcccccaaggtggtcatgactggtctcatttgcagaagtctaagaatgtacctttttctggccgggcgtggtagctcatgcctgtaatcccagcactttgggaggctga

SEQ ID NO: 20 - DNA, 1605 bp, HUMAN
gaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 21 - DNA, 5791 bp, HUMAN
ttcttcctaaatttatttacaaatgtaacacaattccacccaaacttatgtttttataagtaattgagtagatgatcctaaagtttaataaaacaaatggctctaataggtaagacatttggaaatgtataatgaaagggagttgcataataagatcatctatataaatcatctaataaatctacaataaaaagtgtctctagcacagaaataagatatcaatagaatataaggtacaaaatcagattcaggaacattaaagaatatacgacaaaggtgatatttcaagcccaaaggggagaagatggttattcaacacatagtgttttaaaatttgtcagataagaatggagaggaggaggctcctctcctctgaccccagggaatgtgagaagagacacagtggttatgaaaggaagcagtcacacctgtggatccctaccttccccatcagagctagggggcatggagcgctctctgctaagatggggacccccaaggaatgtctccctgtggggcacttccttaccagatgggatggccagtgcggttaagttggtggtcaggcagaaaaaaaagatctagtttgtactcttgagagttcctcggtttgttcatggcatgggcagggagtcaaggagcagcagccttgcctcagtgcctaccagtgcaggaaaaggtgcatagcctgggccagggccagggccctggtggaggcgtagtggtaacagagagggctctccattccagcccaaggaagactaagaatgaatacctcatgagtatattagctacaaaccaccacagcaggttccagaaaaaggctcagcgttggaaccaggtcacccccactcagcagacaccagtcatataaatcaaggaccaacaggagacaggaacacccccttcccactctgccccatgtctcaagttgtagtggcccttcctccagatctctgccaccatcttagaaaggaacactgaaagaagaaactgaaattataagctgacagcataaagaggatgagtaaaacctaaaatcattgttcaaatgaatgaatcaagagaagtttaaaccactttggactaaaatgtgtgaatcctttttcctgctatccagcagatgagaagctggtaacagagaccaaaatagtttggagactaaagaatcattgcacatttcactgctgagttgtattgtgagtaattttagttgacctcacttttgtaaatcttgcacacgggcatccatatctgcacagagatatgttaacagtggtaaatgctgcatgaggagattgggtgatttttactttcgtttttgtgctcttctttcttattgttcttacttatttacgattaccctatcgttttccaaaatgtaaaaggccattttgaaagcctaattcaaacctcttcactattttgtatctaagtattcaccttgattgagactgggtagacaggtgaaaaccatatcaggtttttaattttttaatttttaattatttatttatttatttattttttgagatggagtctggctgtcgcccaggctggagtgcagcggcgtgatcacagttcactgcagcctcaaccttctaggctcaagggattctcccacctcagccccccaagtagttgggaccacacgtatgcgccaccatgcctggctaatttcttatttttttgtagagataggatctcactatattgtccaggctggtcttgaattcctgggctcaggtgagcctcccacctgggcctcccaaagtactgggattacaggcatgagccaaggtcccctgcccatatgagattttctgtctctgatcccatgcagctagtaatcaaggacttggctgctgactctggaggacctgcatgctttcttgagctgtgaacttcagtgctaaaagctcataggcagccctgaaacccaaaccaaaaggttctatggtttatcatcccgatcatgttgattttatagaaataacacatgaattaaagacactaccctcaaactgagcaaaacttaagtaatttttttaaagtttgacctgtttttaaatcactcttggagaaaaaggaaaataaatacaaataattaacggtgaatacaggctactatacctttgttctccagaattagcagttctgttcttttcttgctttagatgctgaagtgcagaaggacactctgtgattgtacgtgtgtaactgacaaaatgtgtattttttttctcagctgctatggattggattatgctattatgaataagaatgctgatgggagcacacacaaaccatttgttcctcagtccattttcctcctcaaaagcctggaatgtgccattgatcagtgggagatgtacctggacagacccatgaaaagagatcaacaagttccacccaagggaccctatttttcctaatttcatttgaaatggcttctaattgtccttctttcattcctgcttcctaccagttttacagctttttctggtttcaaatgtgaactcacatacactctcatttttcctcatcacaaccccaagtgacccaatggtcctcactttcgatataagtaaaggaggctctgcattaagggcttgtccaaggcacgcagctgagaggcgctaggactggctccatttccatctctattctcactgactttgactacccagaaccccaacatgtggggcctcagtattcgatcaattattctattaagaagcaaaaacaattccccgcattggccccagttattaagcatttctcagatttaccttgagaaatgcccatcggcctgtatattcacatcttcacccttgtcccttcctcctagaaaggagaaagtcagttggatgccctctgaggaactagtgcatggcttaactgtccttccatgactcctgccttatctgttttctattttcctccttttccaccgaagtctataatctcaagaaaagcaggcactggccttagggctcctggcctaagaaatatcaagtccagtgagaaatcccattgactgacccctcctgcttacccctttgtgatggagaagctcccaggggtttgctttttgcatgttaccaggcctaactcagcatcaccaggggcaagaaaaggaaagtaacctaaactaatgctgcttataattgtaattattgtaatagttaattactgtgattgtacatgtgtaacagacaaaatgtgtatttttttcacagctgctgtggattggattatgccatttggaataagaatgctgttaagagcacacaagccaggttcctcaagtccgtagcaaatttttcaaaagttaaatttaaaaatcactacatttgaatctagtgacaggagaaatggacatggatagagactaaagatctagcccaaattttatatttacttgttagaggattttgaacaaattactaaatttcttcaaggttcaatttccccattaactataatgaatggctcatcattatggggccctggagaagcataattacttgtaattgtaataatcattgttattattattatacatattttgcttttaaatggataaggatttttaaggtatatgtaaactgtaaaacataaaatgcaaaatgccgtaagagacagtagtaataataatgattattatattgttatcattatctagcctgttttttcctgttttgtatttcttcctttaaatgctttcagaaatctgtatccccattcttcaccaccaccccacaacatttctgcttcttttcccatgccgggtcatgctaactttgaaagcttcagctctttccttcctcaatccttttcctggcacctctgatatgccttttgaaattcatgttaaagaatccctaggctgctatcacatgtggcatctttgttgagtacatgaataaatcaactggtgtgttttacgaaggatgattatgcttcattgtgggattgtatttttcttcttctatcacagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaacataatggcatgccattcctgttctaaatattcttcatagtttgttgatttcctgttcacactgaatgacaaaatttcccatgggtgactcacagagccctgagaagtgtgcactgtcctcttaatgccaatctgacattgcagaggaggccagggcctcctgtacatatgaagtagcccgggtatggagcggggaggtgttgggcttgcagggactctcggcacctgtag

SEQ ID NO: 22 - DNA, 1399 bp, HUMAN
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaat

SEQ ID NO: 23 - DNA, 6919 bp, HUMAN
ttttgcttactacggccagtttctcccgttacataaggccacccccctatctccgcgggccatcgccgccgcaaccgccgcgccagcgccttctcccacgcgcgggggcgcccctgcccaccgctcccggcagggcttttggtggccatgggggataaggggcgttgactcacccgggcggggctccgggagttgcacagaccaaggtagttccccgctccttcccccatcacggagaccctgtgggagatgccgtgggccctctactacagattaggaaacaggcccgtagaggggtcacacggccaagtagcggcactccaggcactgggggccctcgaggggaaggggcagacttctgggagtcagagccagcagctgggctgggaagcttcgagtgtggacagagagggtgggaatgacgttccctgtgggaagagagggtgggcaagcctgggatgcctctgagcgggaatccagcatgccttgtgaggagggtcacaagcacacccttgtgaggaggttgagccccatcgaggacaggacggagggagcctgagcaggcagagagggggcctggggaggcgctggttcggggaggaagtgggtaggggagaaatcttgacatcaacacccaacaggcaaatgccgtggcctctgctgtgggggtttctggaggacttctaggaaaacgagggaagagcaggaaaaggcgacatggctgcaggggccaagcccaggagccgccctccacagcactcattctgcagaagggaaatttgaggcccccagacggcaggggttgatcctgcagagactggtgagcaaaggggatcaccccaagccccagtggcactaggaacacttacaatctctgacctggactaaggctgccagcctggcccagttaagagtttcccagaaggatggcccatacactttaaattaaaggggccagacacgtgcacactacttccagccactctggaagctgaggtggggggatcgcttgagtctgggagttggaggccagcctaggcaggcaacatagtgagaccccatctccaaaaaaacaaaacaaaacaaaacaaaaaaacaccaaaaaagctcccagaaagacctctgaatctttctggatctctcagtggagacctggaaatctgaactttgacaatccctctcacagtggggccaaggaggaattaggcaagccaaaagaagtgaactttactcttctattgcctgtttgaattttgtatccaagcaagtgttacttaagtaatttaagagactggttcatcgaaaaaataaaactccccaaattcccatagctggtagactgtggtcacagccacagtgcactaagactatctgctcagcacttctggtgacccaaaagggtctgaggacaggagctcagagttgggtcagctgtccaggtactcagggttgtcacaggcaaaactgctggaactcagggcagcattgcaaatgccacgccgctctcagggccccttgcctgccgctggaattaaacccacccagatcttggaaactctgccctggacccttctcaataagtccatgagaaatcaaactctttcctttatgcgacactggattttccacaaagtaaaatcaagatgagtaaagatgtggtttctagatagtgcctgaaaaagcagagaccatggtgtcaggcgtcaccacttgggcctataaaagctgccacaagacgccaaggccacaagccacccagcctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggtgagtgtcggctagccagggtcctagctatgagggctccagggtgggtgattcccaagatgaggtcatgagcaggctgggcctggtcctaagatgcctgtaggtcaggaaaaatctccatggaccaaggcccggcccagccatgagggagagaggagctgggctggggggctcagcactgtggatggacctatggaggtgtctggcagactccccagggactacctgctctcctggcctggccttgtctgccactgccagctcctactcagccattcctgaacagaggacagcagagaagggccagcaccctcccagaaccatgtggcatttgccaactggattttgaccataacaatgcagccattctccccagcaccatcataggcccgcccttacaggaggattcgttagtagagtccgctccttgccccactagtaacagctcacatgtctgagcactgcttacaccaggcctggtgcacgtgctttatgtgtcatttcatcactgccagccacctcaagaggcaggtacgatgaacccattctgctaaggttcagtgaggttaagtgacagaggctggattcaagccaggcctggccaacaccagagtgtccatgctcctaactgcagtgttccctcaccatcagaaggcagggcatttaatacaccagatccccaccgcctcccatctgatttgtcttggtcaacagtggcccaggccactcctacttcactcgtccccaccctggcccttcccgcaggcccctgtcctcctgccctgactatggcaagccttgcatgcagcttgtcccttactagtggtgtcaatttttttctctcagctccaagaccctaaacagtgggacctcacccctatgcctgctgttcaaagcagaaaacgaagctcaggaatgctgaggggctgccaggcctgcctctgtgccacaccagggatgcttgtggggcctgtgctggggcagacctggcctgggctgccagggcaggcccacaacccctgccagcactctgctcactgtcactttgctcccacaggctccgctctgcaatggcagcatggtatggagcatcaacctgacagctggcatggtaaggacctttgggtgcagggaggatggggcagaggctccaggccttgggcttatcttctctgagcctcccttccatggctggggttccaagcaagcttcaagtgctctcctccctcccgccataatctggccccttcccgcccaccacccagactcacctgcgccaggcatctcagccccatcttcctgcagactcacaaaaggcagctgcccaagcagggcctgacccctcggtgtcccctccccacagtactgtgcagccctggaatccctgatcaacgtgtcaggctgcagtgccatcgagaagacccagaggatgctgagcggattctgcccgcacaaggtctcagctggggtaaggcatcccccaccctctcacacccaccctgcaccccctcctgccaaccctgggctcgctgaagggaagctggctgaatatccatggtgtgtgtccacccaggggtggggccattgtggcagcagggacgtggccttcgggatttacaggatctgggctcaagggctcctaactcctacctgggcctcaatttccacatctgtacagtagaggtactaacagtacccacctcatggggacttccgtgaggactgaatgagacagtccctggaaagcccctggtttgtgcgagtcgtcccggcctctggcgttctactcacgtgctgacctctttgtcctgcagcagttttccagcttgcatgtccgagacaccaaaatcgaggtggcccagtttgtaaaggacctgctcttacatttaaagaaactttttcgcgagggacggttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctggcacttggagccaagggttcagagactcagggccccagcactaaagcagtggaccccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttcctcgtatttaaatattaaatatgttagcaaagagttaatatatagaagggtaccttgaacactgggggaggggacattgaacaagttgtttcattgactatcaaactgaagccagaaataaagttggtgacagataggcctgattgtatttgtctttcattttggcctttggggacactggtctgtggtctgaagactctgaggagctcttcgggaggctggtgggttggaggaggggactgggatggattacagcgagggtagggtgcagtgacctgggctgaatgcaagctagctcccgagggtggggacatggcctgaaggaagccccaccttctgtctgctgcaccagcaaggacggagaggcttgggccagactgtcagggttcaaggagggcatcaggagcagacggagacccaggaagtctcacaatcacatctcctgaggactggccagctgtgtctggcaccacccacacatccatgtctccctcacaacccaggaggccgatgagaactgtgaggctcagaaagcgtgggcggtttgcctaaggtcacgtagctacttcctcactggggtcctggggcctcagagcctcatctgaggtaaaggagcaaagttgggattggggtccaaaattcactttaactccaaagcccacacacttaaccaccctgcctatttctgtccaaatgtcacctgtcctgaatggagtttttccccctgtacaactgtcatcaacctgttcgggccctctcactgacaggcaggtccctacctatatttgaggggcagcccattgcatttctggacagctctcgccattagggtgcacacacgcaccacctctgtgaacagggctctggctaggccactcctcagcagctcttgttgcttccccatggccctggtcagcagctggagtgcagagaccagcgggccttaccaagccacagctccaggccatgcccgtcagcaacacttttcactgtgactctctgggaggtgcccagggcagagggtgactccaggatgggatgcctttgcagtgggtgatggtcttcaagaaccagtctcaaacttgtagtgacaggcaggtgccctccctggggagcccagagccctatgggaacaccacagtgatgctcagaagtccctgaaggcagccgccctcgactgctcatggctgtgtcattcacgcaacagcagaggacgtggcagggagttcgccagggggctgatgatggtggccagctactccagaaccactcactttccccagggagaggcgacggaagaaggcaaacctgacagtgcagggggcaggggacacttggcctagaggagagaagactgtgggggtgaagagggtgatggtgcaagagctgggtctccagcaggctgtcccgggcaggagacaggaccggctctctggtgccaagcctattaagtcctgccacgtttgctgaggaaatacctgcgggggaccaccccacagcagacacctgggctagtgacaaccaagacaccacatgcactcaagatttagcccctgaaagaactctgtagaaacaacagagggtcgggttgggggagctgtatgtcctccctcgggagggcctcccctctggaggtgagcagtggctgcttccgcaaaggacgagagagtcttggaagccaggggaggtgggtgggagtcagggtctcggcattcttctccatccagagcctagggtttaaggctggaaggatatttacggagggtaatgactttgtaaacaagacctagaaatctatctcccagtagcatcatctttgacccatgtcctctctgcccagccaaagtattgccctgcctatccagaaagatggaaacccttccagtgaaattccacgtgactccctcaagaacaaagactggaattgaggctgatgtggggcaaggttcctcgggatcctccaaggccgtgctctgagccacacattcaggacagcctgcgaggcaggtgcacagtgcggatggccgtcccgggccccactaggggtctgcagctcgccacttcctcagccaccgccgagctcctgtccctcacaccttccaggctgctccccaggaagggggacctgtggtcactggcagtgtgccctgggctggagagggcgtggggctctccagggatgagtgacaccttactgagccacagaaccgagctgcagctggtggcgacagggaaggggccctgtcaggaatgagaaaggaagacagaacaaggtaggtaagtgtcagcactgcggtcatagctgttttccctggggagggggaaagggccagcccttccggcattggcctgtttccctgagggggggg

SEQ ID NO: 24 - DNA, 1610 bp, HUMAN
cccagagcagcgctcgccacctccccccggcctgggcagcgctcgcccggggagtccagcggtgtcctgtggagctgccgccatggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgaaactcggggaaaccagcccagctaagtccggagtgaaggagcctctctgctttagctaaagacgactgagaagaggtgcaaggaagcgggctccaggagcaagctcaccaggcctctcagaagtcccagcaggatctcacggactgccgggtcggcgcctcctgcgcgagggagcaggttctccgcattcccatgggcaccacctgcctgcctgtcgtgccttggacccagggcccagcttcccaggagagaccaaaggcttctgagcaggatttttatttcattacagtgtgagctgcctggaatacatgtggtaatgaaataaaaaccctgccccgaatcttccgtccctcatcctaactttcagttcacagagaaaagtgacatacccaaagctctctgtcaattacaaggcttctcctggcgtgggagacgtctacagggaagacaccagcgtttgggcttctaaccaccctgtctccagctgctctgcacacatggacagggacctgggaaaggtgggagagatgctgagcccagcgaatcctctccattgaaggattcaggaagaagaaaactcaactcagtgccattttacgaatatatgcgtttatatttatacttccttgtctattatatctatacattatatattatttgtattttgacattgtaccttgtataaacaaaataaaacatctattttcaatatttttaaaatgca

SEQ ID NO: 25 - DNA, 3567 bp, HUMAN
cgccatcccgcgctctgcggactgggaggcccgggccaggacgcgagtctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctcggcggcagaaaccgggagtggggccgggcgagtgcgcggcatcccaggccggcccgaacgtccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgcttagtgttgaatgttccccaccgagagcgcatggcttgggaagcgaggcgcgaacccgggccccgaagccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggatccatcgtgacttgtgggcatagatggaaaaatatattttacataaagaatgaaaataagctccccactggtggttgctatggagtgccccctgatttacgaacagaactgagtaaaagaatagctccgtgttatcaagattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttaggatattcagtcggagctggtcattttcggagccagcatactaccgaagtagtcggaggagctcctcaacatgagcagattggtaaggcatatatattcagcattgatgaaaaagaactaaatatcttacatgaaatgaaaggtaaaaagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaagatgttgctatcggagctccacaagaagatgacttgcaaggtgctatttatatttacaatggccgtgcagatgggatctcgtcaaccttctcacagagaattgaaggacttcagatcagcaaatcgttaagtatgtttggacagtctatatcaggacaaattgatgcagataataatggctatgtagatgtagcagttggtgcttttcggtctgattctgctgtcttgctaaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattgttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcggaaagatgtgcgggacatcctcaccccaattcagattgaagctgcttaccaccttggtcctcatgtcatcagtaaacgaagtacagaggaattcccaccacttcagccaattcttcagcagaagaaagaaaaagacataatgaaaaaaacaataaactttgcaaggttttgtgcccatgaaaattgttctgctgatttacaggtttctgcaaagattgggtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccagttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcatgttctactggaaggactacatcatcaaagacccaaacgttatttcaccatagtgattatttcaagtagcttgctacttggacttattgtacttctgttgatctcatatgttatgtggaaggctggcttctttaaaagacaatacaaatctatcctacaagaagaaaacagaagagacagttggagttatatcaacagtaaaagcaatgatgattaaggacttctttcaaattgagagaatggaaaacagcccgccc

SEQ ID NO: 26 - DNA, 2477 bp, HUMAN
agtgcagacgcggctcctagcggatgggtgctattgtgaggcggttgtagaagttaataaaggtatccatggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaaagaaatggttggttggtggttttttttagtttgtatgccaagtgagaagatggtatatttggtactgtatttccctctcattttgacctactctcatgctgcagagggtactttaagacatactccttccatcaaatagaaccactatgaagctacctcaaacttccagtcaggtagttgcaattgaattaaattaggaataaataaaaatggatactggtgcagtcattatgagaggcaatgattgttaatttacagctttcatgattagcaagttacagtgatgctgtgctatgaattttcaagtaattgtgaaaaagttaaacattgaagtaatgaatttttatgatattccccccacttaagactgtgtattctagttttgtcaaactgtagaaatgatgatgtggaagaacttaggcatctgtgggcatggtcaaaggctcaaacctttattttagaattgatatacacggatgacttaactgcatttttagaccatttatctgggattatggttttgtgatgtttgtcctgaacacttttgttgtaaaaaaataataataatgtttaatattgagaaagaaactaatattttatgtgagagaaagtgtgagcaaactaacttgacttttaaggctaaaacttaacattcatagaggggtggagttttaactgtaaggtgctacaatgcccctggatctaccagcataaatatcttctgatttgtccctatgcatatcagttgagcttcatataccagcaatatatctgaagagctattatataaaaaccccaaactgttgattattagccaggtaatgtgaataaattctataggaacatatgaaaatacaacttaaataataaacagtggaatataaggaaagcaataaatgaatgggctgagctgcctgtaacttgagagtagatggtttgagcctgagcagagacatgactcagcctgttccatgaaggcagagccatggaccacgcaggaagggcctacagcccatttctccatacgcactggtatgtgtggatgatgctgccagggcgccatcgccaagtaagaaagtgaagcaaatcagaaacttgtgaagtggaaatgttctaaaggtggtgaggcaataaaaatcatagtactctttgtagcaaaattcttaagtatgttattttctgttgaagtttacaatcaaaggaaaatagtaatgttttatactgtttactgaaagaaaaagacctatgagcacataggactctagacggcatccagccggaggccagagctgagccctcagcccgggaggcaggctccaggcctcagcaggtgcggagccgtcactgcaccaagtctcactggctgtcagtatgacatttcacgggagatttcttgttgctcaaaaaatgagctcgcatttgtcaatgacagtttcttttttcttactagacctgtaacttttgtaaatacacatagcatgtaatggtatcttaaagtgtgtttctatgtgacaattttgtacaaatttgttattttccatttttatttcaaaatatacattcaaacttaaaatt

SEQ ID NO: 27 - DNA, 3199 bp, HUMAN
gtttttctaaacagcctgacactgaggggaggcagtgagactgtaagcagtctgggttgggcagaaggcagaaaaccagcagagtcacagaggagatggccaactgccaaatagccatcttgtaccagagattccagagagtggtctttggaatttcccaactcctttgcttcagtgccctgatctctgaactaacaaaccagaaagaagtggcagcatggacttatcattacagcacaaaagcatactcatggaatatttcccgtaaatactgccagaatcgctacacagacttagtggccatccagaataaaaatgaaattgattacctcaataaggtcctaccctactacagctcctactactggattgggatccgaaagaacaataagacatggacatgggtgggaaccaaaaaggctctcaccaacgaggctgagaactgggctgataatgaacctaacaacaaaaggaacaacgaggactgcgtggagatatacatcaagagtccgtcagcccctggcaagtggaatgatgagcactgcttgaagaaaaagcacgcattgtgttacacagcctcctgccaggacatgtcctgcagcaaacaaggagagtgcctcgagaccatcgggaactacacctgctcctgttaccctggattctatgggccagaatgtgaatacgtgagagagtgtggagaacttgagctccctcaacacgtgctcatgaactgcagccaccctctgggaaacttctcttttaactcgcagtgcagcttccactgcactgacgggtaccaagtaaatgggcccagcaagctggaatgcttggcttctggaatctggacaaataagcctccacagtgtttagctgcccagtgcccacccctgaagattcctgaacgaggaaacatgatctgccttcattctgcaaaagcattccagcatcagtctagctgcagcttcagttgtgaagagggatttgcattagttggaccggaagtggtgcaatgcacagcctcgggggtatggacagccccagccccagtgtgtaaagctgtgcagtgtcagcacctggaagcccccagtgaaggaaccatggactgtgttcatccgctcactgcttttgcctatggctccagctgcaaatttgagtgccagcccggctacagagtgaggggcttggacatgctccgctgcattgactctggacactggtctgcacccttgccaacctgtgaggctatttcgtgtgagccgctggagagtcctgtccacggaagcatggattgctctccatccttgagagcgtttcagtatgacaccaactgtagcttccgctgtgctgaaggtttcatgctgagaggagccgatatagttcggtgtgataacttgggacagtggacagcaccagccccagtctgtcaagctttgcagtgccaggatctcccagttccaaatgaggcccgggtgaactgctcccaccccttcggtgcctttaggtaccagtcagtctgcagcttcacctgcaatgaaggcttgctcctggtgggagcaagtgtgctacagtgcttggctactggaaactggaattctgttcctccagaatgccaagccattccctgcacacctttgctaagccctcagaatggaacaatgacctgtgttcaacctcttggaagttccagttataaatccacatgtcaattcatctgtgacgagggatattctttgtctggaccagaaagattggattgtactcgatcgggacgctggacagactccccaccaatgtgtgaagccatcaagtgcccagaactctttgccccagagcagggcagcctggattgttctgacactcgtggagaattcaatgttggctccacctgtcatttctcttgtaacaatggctttaagctggaggggcccaataatgtggaatgcacaacttctggaagatggtcagctactccaccaacctgcaaaggcatagcatcacttcctactccagggttgcaatgtccagccctcaccactcctgggcagggaaccatgtactgtaggcatcatccgggaacctttggttttaataccacttgttactttggctgcaacgctggattcacactcataggagacagcactctcagctgcagaccttcaggacaatggacagcagtaactccagcatgcagagctgtgaaatgctcagaactacatgttaataagccaatagcgatgaactgctccaacctctggggaaacttcagttatggatcaatctgctctttccattgtctagagggccagttacttaatggctctgcacaaacagcatgccaagagaatggccactggtcaactaccgtgccaacctgccaagcaggaccattgactatccaggaagccctgacttactttggtggagcggtggcttctacaataggtctgataatgggtgggacgctcctggctttgctaagaaagcgtttcagacaaaaagatgatgggaaatgccccttgaatcctcacagccacctaggaacatatggagtttttacaaacgctgcatttgacccgagtccttaaggtttccataaacacccatgaatcaaagacatggaattaccttagattagctctggaccagcctgttggacccgctctggaccaaccctgtttcctgagtttgggattgtggtacaatctcaaattctcaacctaccaccccttcctgtcccacctcttctcttcctgtaacacaagccacagaagccaggagcaaatgtttctgcagtagtctctgtgctttgactcacctgttacttgaaataccagtgaaccaaagagactggagcatctgactcacaagaagaccagactgtggagaaataaaaatacctctttattttttgattgaaggaaggttttctccactttgttggaaagcaggtggcatctctaattggaagaaattcctgtagcatcttctggagtctccagtggttgctgttgatgaggcctcttggacctctgctctgaggcttccagagagtcctctggatggcaccagaggctgcagaaggccaagaatcaagctagaaggccacatgtcaccgtggaccttcctgccaccagtcactgtccctcaaatgacccaaagaccaatattcaaatgcgtaattaaaagaattttcccc

SEQ ID NO: 28 - DNA, 5133 bp, HUMAN
cctctttcaccctgtctaggttgccagcaaatcccacgggcctcctgacgctgcccctggggccacaggtccctcgagtgctggaaggatgaaggattcctgcatcactgtgatggccatggcgctgctgtctgggttctttttcttcgcgccggcctcgagctacaacctggacgtgcggggcgcgcggagcttctccccaccgcgcgccgggaggcactttggataccgcgtcctgcaggtcggaaacggggtcatcgtgggagctccaggggaggggaacagcacaggaagcctctatcagtgccagtcgggcacaggacactgcctgccagtcaccctgagaggttccaactatacctccaagtacttgggaatgaccttggcaacagaccccacagatggaagcattttggcctgtgaccctgggctgtctcgaacgtgtgaccagaacacctatctgagtggcctgtgttacctcttccgccagaatctgcagggtcccatgctgcaggggcgccctggttttcaggaatgtatcaagggcaacgtagacctggtatttctgtttgatggttcgatgagcttgcagccagatgaatttcagaaaattctggacttcatgaaggatgtgatgaagaaactcagcaacacttcgtaccagtttgctgctgttcagttttccacaagctacaaaacagaatttgatttctcagattatgttaaatggaaggaccctgatgctctgctgaagcatgtaaagcacatgttgctgttgaccaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacatttgagaagctgaaagatctattcactgagctgcagaagaagatctatgtcattgagggcacaagcaaacaggacctgacttccttcaacatggagctgtcctccagcggcatcagtgctgacctcagcaggggccatgcagtcgtgggggcagtaggagccaaggactgggctgggggctttcttgacctgaaggcagacctgcaggatgacacatttattgggaatgaaccattgacaccagaagtgagagcaggctatttgggttacaccgtgacctggctgccctcccggcaaaagacttcgttgctggcctcgggagcccctcgataccagcacatgggccgagtgctgctgttccaagagccacagggcggaggacactggagccaggtccagacaatccatgggacccagattggctcttatttcggtggggagctgtgtggcgtcgacgtggaccaagatggggagacagagctgctgctgattggtgccccactgttctatggggagcagagaggaggccgggtgtttatctaccagagaagacagttggggtttgaagaagtctcagagctgcagggggaccccggctacccactcgggcggtttggagaagccatcactgctctgacagacatcaacggcgatgggctggtagacgtggctgtgggggcccctctggaggagcagggggctgtgtacatcttcaatgggaggcacggggggcttagtccccagccaagtcagcggatagaagggacccaagtgctctcaggaattcagtggtttggacgctccatccatggggtgaaggaccttgaaggggatggcttggcagatgtggctgtgggggctgagagccagatgatcgtgctgagctcccggcccgtggtggatatggtcaccctgatgtccttctctccagctgagatcccagtgcatgaagtggagtgctcctattcaaccagtaacaagatgaaagaaggagttaatatcacaatctgtttccagatcaagtctctctacccccagttccaaggccgcctggttgccaatctcacttacactctgcagctggatggccaccggaccagaagacgggggttgttcccaggagggagacatgaactcagaaggaatatagctgtcaccaccagcatgtcatgcactgacttctcatttcatttcccggtatgtgttcaagacctcatctcccccatcaatgtttccctgaatttctctctttgggaggaggaagggacaccgagggaccaaagggcgcagggcaaggacataccgcccatcctgagaccctccctgcactcggaaacctgggagatcccttttgagaagaactgtggggaggacaagaagtgtgaggcaaacttgagagtgtccttctctcctgcaagatccagagccctgcgtctaactgcttttgccagcctctctgtggagctgagcctgagtaacttggaagaagatgcttactgggtccagctggacctgcacttccccccgggactctccttccgcaaggtggagatgctgaagccccatagccagatacctgtgagctgcgaggagcttcctgaagagtccaggcttctgtccagggcattatcttgcaatgtgagctctcccatcttcaaagcaggccactcggttgctctgcagatgatgtttaatacactggtaaacagctcctggggggactcggttgaattgcacgccaatgtgacctgtaacaatgaggactcagacctcctggaggacaactcagccactaccatcatccccatcctgtaccccatcaacatcctcatccaggaccaagaagactccacactctatgtcagtttcacccccaaaggccccaagatccaccaagtcaagcacatgtaccaggtgaggatccagccttccatccacgaccacaacatacccaccctggaggctgtggttggggtgccacagcctcccagcgaggggcccatcacacaccagtggagcgtgcagatggagcctcccgtgccctgccactatgaggatctggagaggctcccggatgcagctgagccttgtctccccggagccctgttccgctgccctgttgtcttcaggcaggagatcctcgtccaagtgatcgggactctggagctggtgggagagatcgaggcctcttccatgttcagcctctgcagctccctctccatctccttcaacagcagcaagcatttccacctctatggcagcaacgcctccctggcccaggttgtcatgaaggttgacgtggtgtatgagaagcagatgctctacctctacgtgctgagcggcatcggggggctgctgctgctgctgctcattttcatagtgctgtacaaggttggtttcttcaaacggaacctgaaggagaagatggaggctggcagaggtgtcccgaatggaatccctgcagaagactctgagcagctggcatctgggcaagaggctggggatcccggctgcctgaagcccctccatgagaaggactctgagagtggtggtggcaaggactgagtccaggcctgtgaggtgcagagtgcccagaactggactcaggatgcccagggccactctgcctctgcctgcattctgccgtgtgccctcgggcgagtcactgcctctccctggccctcagtttccctatctcgaacatggaactcattcctgaatgtctcctttgcaggctcatagggaagacctgctgagggaccagccaagagggctgcaaaagtgagggcttgtcattaccagacggttcaccagcctctcttggttccttccttggaagagaatgtctgatctaaatgtggagaaactgtagtctcaggacctagggatgttctggccctcacccctgccctgggatgtccacagatgcctccaccccccagaacctgtccttgcacactcccctgcactggagtccagtctcttctgctggcagaaagcaaatgtgacctgtgtcactacgtgactgtggcacacgccttgttcttggccaaagaccaaattccttggcatgccttccagcaccctgcaaaatgagaccctcgtggccttccccagcctcttctagagccgtgatgcctccctgttgaagctctggtgacaccagcctttctcccaggccaggctccttcctgtcttcctgcattcacccagacagctccctctgcctgaaccttccatctcgcccacccctccttccttgaccagcagatcccagctcacgtcacacacttggttgggtcctcacatctttcacacttccaccaccctgcactactccctcaaagcacacgtcatgtttcttcatccggcagcctggatgttttttccctgtttaatgattgacgtacttagcagctatctctcagtgaactgtgagggtaaaggctatacttgtcttgttcaccttgggatgacgccgcatgatatgtcagggcgtgggacatctagtaggtgcttgacataatttcactgaattaatgacagagccagtgggaagatacagaaaaagagggccggggctgggcgcggtggttcacgcctgtaatcccagcactttgggaggccaaggagggtggatcacctgaggtcaggagttagaggccagcctggcgaaaccccatctctactaaaaatacaaaatccaggcgtggtggcacacacctgtagtcccagctactcaggaggttgaggtaggagaattgcttgaacctgggaggtggaggttgcagtgagccaagattgcgccattgcactccagcctgggcaacacagcgagactccgtctcaaggaaaaaataaaaataaaaagcgggcacgggcccggacatccccacccttggaggctgtcttctcaggctctgccctgccctagctccacaccctctcccaggacccatcacgcctgtgcagtggcccccacagaaagactgagctcaaggtgggaaccacgtctgctaacttggagccccagtgccaagcacagtgcctgcatgtatttatccaataaatgtgaaattctgtccaaaaaaaaaaaa

SEQ ID NO: 29 - DNA, 1172 bp, HUMAN
gagacattcctcaattgcttagacatattctgagcctacagcagaggaacctccagtctcagcaccatgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatattaagaaagactatctaaatgttgaaagatcaaaaggttaataaagtaattataact

SEQ ID NO: 30 - DNA, 1497 bp, HUMAN
accaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttctctgaag

SEQ ID NO: 31 - DNA, 808 bp, HUMAN
tgctcccttgggctctagagaggaggcccctcttagccctcagcccctccttcctctctatcttaaagtaatttgatcctcaggaatttgttccgccctcatctggcccggccaaatcccgatttgacaaatgccaggaaaaggaaactgttgagaaaccgaaactactggggaaagggagggctcactgagtaaccatcccagtaacccgaccgccgctggtcttcgctggacaccatgagtcacactgtccaaaccttcttctctcctgtcaacagtggccagccccccaactatgagatgctcaaggaggagcacgaggtggctgtgctgggggggccccacaaccctgctcccccgacgtccaccgtgatccacatccgcagcgagacctccgtgcccgaccatgtcgtctggtccctgttcaacaccctcttcatgaacccctgctgcctgggcttcatagcattcgcctactccgtgaagtctagggacaggaagatggttggcgacgtgaccggggcccaggcctatgcctccaccgccaagtgcctgaacatctgggccctgattctgggcatcctcatgaccattctgctcatcgtcatcccagtgctgatcttccaggcctatggatagatcaggaggcatcactgaggccaggagctctgcccatgacctgtatcccacgtactccaacttccattcctcgccctgcccccggagccgagtcctgtatcagccctttatcctcacacgcttttctacaatggcattcaataaagtgcacgtgtttctggtgctgctg

SEQ ID NO: 32 - DNA, 905 bp, HUMAN
caacacaggggcagtctccaggacctccacaccattaacaagatgagccttgtgctcccttgggctctagagaggaagcccctctgagccctcagcccctctttcctccctctcctaaagtaatttgatcctcaggaatttgttctgccctcatctggccctggccagctctgcatttgacaaatgccaggaagaggaaactgttgagaaaacggaactactggggaaagggagggctcactgagaaccatcccggtaacccgaccgccgctggtcaccatgaaccacattgtgcaaaccttctctcctgtcaacagcggccagcctcccaactacgagatgctcaaggaggagcaggaagtggctatgctgggggggccccacaaccctgctcccccgacgtccaccgtgatccacatccgcagcgagacctccgtgcctgaccatgtcgtctggtccctgttcaacaccctcttcatgaacacctgctgcctgggcttcatagcattcgcctactccgtgaagtctagggacaggaagatggttggcgacgtgaccggggcccaggcctatgcctccaccgccaagtgcctgaacatctgggccctgattttgggcatcttcatgaccattctgctcgtcatcatcccagtgttggtcgtccaggcccagcgatagatcaggaggcatcattgaggccaggagctctgcccgtgacctgtatcccacgtactctatcttccattcctcgccctgcccccagaggccaggagctctgcccttgacctgtattccacttactccaccttccattcctcgccctgtccccacagccgagtcctgcatcagccctttatcctcacacgcttttctacaatggcattcaataaagtgtatatgtttct

SEQ ID NO: 33 - DNA, 3444 bp, HUMAN
atggcttgcccctggaagtttctcttcagagtcaaatcctaccaaggtgacctgaaagaggaaaaggacattaacaacaacgtggagaaaaccccaggtgctattcccagcccaacaacacaggatgaccctaagagtcacaagcatcaaaatggtttcccccagttcctcactgggactgcacagaatgttccagaatccctggacaagctgcatgtgactccatcgacccgcccacagcacgtgaggatcaaaaactggggcaatggagagatttttcacgacacccttcaccacaaggccacctcggatatctcttgcaagtccaaattatgcatggggtccatcatgaactccaagagtttgaccagaggacccagagacaagcccaccccagtggaggagcttctgcctcaagccattgaattcattaaccagtattatggctccttcaaagaggcaaaaatagaggaacatctggccaggctggaagccgtaacaaaggaaatagaaacaacaggaacctaccagctcactctggatgagctcatctttgccaccaagatggcctggaggaacgcccctcgctgcatcggcaggattcagtggtccaacctgcaggtcttcgatgcccggagctgtagcactgcatcagaaatgttccagcatatctgcagacacatactttacgccactaacagtggcaacatcaggtcggccattactgtgttcccccagcggaacgatgggaagcatgacttccggatctggaattcccagctcatccggtacgctggctaccagatgcccgatggcaccatcagaggggatcctgccaccttggagttcacccagttgtgcatcgacctgggctggaagccccgctatggccgcttcgatgtgctgcctctggtcctgcaggctcacggtcaagatccagaggtctttgaaatccctcctgatcttgtgctggaggtgaccatggagcatcccaagtacgagtggttccaggagctcgggctgaagtggtatgcgctgcctgccgtggccaacatgctcctggaggtgggtggcctcgagttcccagcctgccccttcaatggttggtacatgggcaccgagattggagtccgagacctctgtgacacacagcgctacaacatcctggaggaagtgggcaggaggatgggcctggggacccacacactggcctccctctggaaagaccgggctgtcaccgagatcaatgcagctgtgctccatagttttcagaagcagaatgtgaccatcatggaccaccacacagcctcagagtccttcatgaagcacatgcagaatgagtaccgggcccgaggaggctgccctgcagactggatttggctggtccctccggtgtccgggagcatcacccctgtgttccaccaggagatgttgaactacgtcctatctccattctactactaccagatcgagccctggaagacccacatctggcaggatgagaagctgaggcccaggaggagagagatccggttcacagtcttggtgaaagcggtgttctttgcttctgtgctaatgcggaaggtcatggcttcccgcgtcagagccacagtcctctttgctactgagacaggaaagtcggaagcgctagccagggacctggctgccttgttcacgtacgccttcaacaccaaggttgtctgcatggaacagtataaggcaaacaccttggaagaggaacaactactgctggtggtgacaagcacatttggcaatggagactgccccagcaatgggcagactctgaagaaatctctgttcatgatgaaagaactcgggcataccttcaggtatgcggtatttggcctgggctccagcatgtaccctcagttctgtgcctttgctcatgacatcgacccgaaactgtctcacctgggagcctcccagcttgccccaaccggagaaggggacgaactcagcgggcaggaggacgccttccgcagctgggctgtgcaaaccttccgggcagcctgtgagacgttcgatgttcgaagcaaacattgcattcagatcccgaaacgctacacttccaacgcaacatgggagccagagcagtacaagctcacccagagcccagagcctctagacctcaacaaagctctcagcagcatccacgccaagaacgtgttcgccatgaggctgaaatccctccagaatctgcagagtgagaagtccagccgcaccaccctccttgttcaactcaccttcgagggcagccgaggccccagctacctacctggggaacacctggggattttcccaggcaaccagacggccctggtgcaagggatcttggagcgagttgtggattgttcttcgccagaccaaactgtgtgcctggaagttctagatgagagtggcagctactgggtcaaagacaagaggcttcccccctgttcactcaggcaagccctcacctacttcctggacatcactacccctcccacccagctgcagctccacaagctggcccgctttgccacggaagagacgcacaggcagaggttggaggccttgtgtcagccctcagagtacaacgattggaagttcagcaacaaccccacgttcctggaggtgctggaagagttcccatcattgcgtgtgcctgctgccttcctgctgtcgcagctccccattctgaagccccgctactactccatcagctcctcccaggaccacaccccctcggaggtccacctcactgtggctgtggtcacctatcgcacccgagatggtcagggtcccctgcaccatggcgtctgcagcacttggatcaataacctgaagcccgaagacccagtgccctgctttgtgcggagtgtcagtggcttccagctccctgaggacccctcccagccctgcatcctcattgggcccggtacaggcattgcccccttccgaagtttctggcagcagcggctccatgactctcagcgcagagggctcaaaggaggccgcatgaccttggtgtttgggtgcagacacccagaggaggaccacctctatcaggaagaaatgcaggagatggtccgcaagggagtgttgttccaggtgcacacaggctactcccggctgcccggaaaacccaaggtctacgttcaagacatcctgcagaaagagctggccgacgaggtgttcagcgtgctccacggggagcagggccacatctatgtttgtggcgatgtgcgcatggctcgggatgtggctaccactttgaagaagctggtggccgccaagctgaacttgagtgaggagcaggttgaggattacttcttccagctcaagagccagaaacgttatcatgaggatatcttcggtgcggtcttttcctatggagtgaaaaagggcaacgccttggaggagcccaaaggcacaagactctga

SEQ ID NO: 34 - DNA, 1186 bp, HUMAN
ggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcaaaaaaaaaaaaaaaaaaaaaaaaaaaaaaaaaaaaaaaaaaaac

(19) United States
(12) Patent Application Publication
(10) Pub. No.: US 2005/0064483 A1
(43) Pub. Date: Mar. 24, 2005

(54) Gene expression profiling technology for treatment evaluation of multiple sclerosis

(75) Inventors: Jingwu Z. Zang, Missouri City, TX (US); Jian Hong, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 10/929,182

(22) Filed: Aug. 30, 2004

Related U.S. Application Data
(60) Provisional application No. 60/498,731, filed on Aug. 28, 2003.

(51) Int. Cl. C12Q 1/68

(52) U.S. Cl. 435/6

(57) ABSTRACT

The invention relates to gene expression profiling technology to quantitatively measure the expression profiles of genes selected based on their role in inflammation and their susceptibility to regulation by current multiple sclerosis (MS) treatment agents, beta-interferon (IFN) and glatiramer acetate (GA). The invention also provides an assay for detection of beta-IFN neutralizing antibody based on the blocking effect of serum antibodies on the known regulatory properties of beta-IFN on PBMC and evaluation of treatment responses in MS patients.

### CROSS-REFERENCE TO RELATED APPLICATIONS

This application claims priority to U.S. Provisional Application No. 60/498,731, filed on Aug. 28, 2003.

### STATEMENT REGARDING FEDERALLY SPONSORED RESEARCH OR DEVELOPMENT

The present invention was developed with funds from the United States Government grant number NS14239. Therefore, the United States Government may have certain rights in the invention.

### TECHNICAL FIELD

The field of the invention relates to molecular biology, genetics, and medicine. The present invention also relates to the field of multiple sclerosis and gene expression profiling.

### BACKGROUND OF THE INVENTION

Multiple sclerosis (MS) is a chronic inflammatory and demyelinating disease of the central nervous system. There is increasing evidence indicating MS is associated with autoimmune inflammation involving activation and aberrant trafficking of T cells and other inflammatory cells which produce an array of inflammatory molecules (e.g. cytokines, chemokines, their receptors and molecules related to T cell adhesion, trafficking and apoptosis) (Ahmed et al., 2002; Cannella and Raine, 1995; Sorensen et al., 1999; Strunk et al., 2000; Zipp et al., 1998b). The production of these molecules not only characteristically reflects the in vivo activity of inflammatory cells but also has clinical relevance to disease activity in MS. There are indications that the changes in some of these serum inflammatory molecules correlate with brain lesion activity as measured by magnetic resonance imaging (MRI) as well as clinical progression in MS (Adachi et al., 1990; Balashov et al., 1997; de Jong et al., 2002; Fazekas et al., 2001; Killestein et al., 2001; Lee et al., 1999; Rieckman et al., 1995; Sharief and Hentges, 1991; van Boxel-Dezaire et al., 1999). It is hoped that these clinically relevant inflammatory molecules may serve as sensitive biomarkers for monitoring disease progression in MS. A simple and sensitive bioassay capable of measuring clinically relevant biomarkers is a pressing need for the evaluation of current MS treatments that include beta-interferons (beta-IFN) and Glatiramer Acetate (GA). Both drugs are known to act as immunomodulatory agents that regulate the activation and migration of inflammatory T cells as well as their ability to produce certain pro- and anti-inflammatory cytokines and chemokines (Galboiz et al., 2001; Hua et al., 1998; Koike et al., 2003; Rudick et al., 1996; Wandinger et al., 2001; Weinstock-Guttman et al., 2003; Yong et al., 1998). These immunomodulatory properties of beta-IFN and GA are thought to contribute to the treatment effect in MS (The IFNB Multiple Sclerosis Study Group, 1993; Jacobs et al., 1996; Johnson et al., 1995; Zhang et al., 2002). Studies have revealed that immunologic alterations induced by beta-IFN or GA treatment are characteristic for certain biomarkers. For example, beta-IFN up-regulates IL-10 and inhibits TNFa and matrix metaloproteinase (MMP)-9, a critical molecule for T cell trafficking (Galboiz et al., 2001; Leppert et al., 1996; Rudick et al., 1996), while GA characteristically induces Th2 immune reaction by activating Th2 cells (Duda et al., 2000; Gran et al., 2000; Neuhaus et al., 2000). The findings have demonstrated that analysis of a set of biomarkers that are relevant to inflammatory processes involved in MS and that are characteristically regulated by beta-IFN may help to assist evaluation and management of MS treatments.

The potential application of measuring relevant biomarkers is particularly important for many clinical situations because progression of MS is relatively insensitive to standard clinical measures. Although advanced magnetic resonance imaging (MRI) technology represents a suitable research tool to assess the activity of the CNS pathology, its routine and frequent utility for treatment monitoring in MS is limited. Furthermore, there is considerable variability in individual responses of MS patients to the drugs (The IFNB Multiple Sclerosis Study Group, 1993; Jacobs et al., 1996; Sturzebecher et al., 2003). Some patients respond to beta-IFN but not GA, or vice versa. It has been difficult to evaluate in a timely fashion the treatment effect of both beta-IFN and GA in MS patients because of the slowly progressive nature of the disease and because of the low sensitivity of current clinical measurements. For both beta-IFN and GA, it often takes 3-9 months before clinical effects become measurable in patients that respond to the treatments (The IFNB Multiple Sclerosis Study Group, 1993; PRISMS Study Group, 1998; Comi et al., 2001; Jacobs et al., 1996). As a result, the valuable treatment time window may be lost in certain clinical situations. These problems are further complicated by the development of neutralizing antibodies in a significant proportion of patients treated with beta-IFN, which also contributes to the loss of clinical benefit (The IFNB Multiple Sclerosis Study Group and the University of British Columbia MS/MRI Analysis Group, 1996).

Conceivably, a bioassay capable of profiling a collective array of biomarkers that are both relevant to MS disease activity and susceptible to the immunoregulatory effects of beta-IFN or GA may provide a more sensitive and useful tool to assist monitoring of current MS treatments.

### BRIEF SUMMARY OF THE INVENTION

An embodiment of the present invention is a method of monitoring a multiple sclerosis patient taking β-interferon comprising the steps of: obtaining a sample of peripheral blood mononuclear cells from the patient; isolating RNA from the sample; and determining the relative expression profile in the isolated RNA of at least four individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and comparing the relative expression profile of the individual nucleic acids to a control sample, wherein the beta-IFN is predicted to be therapeutically effective if the relative expression profile is characteristic of a beta-IFN therapy response.

In a specific embodiment, determining the relative expression of individual nucleic acids in the RNA comprises the steps of: providing a plurality of probes bound to a solid surface, at least four of said plurality of probes being complementary to sequences selected from the group of nucleic acids consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; contacting the probes with the RNA obtained from the sample of peripheral blood mononuclear cells; and detecting binding of the RNA to the probes; thereby identifying differences in relative expression of the nucleic acids.

In a further specific embodiment, the detecting of binding comprises detecting fluorescent or radioactive labels. In yet another embodiment, the solid surface is glass or nitrocellulose.

In one embodiment, at least one of the individual nucleic acids is selected from the group consisting of SEQ ID NO:15, SEQ ID NO:29, SEQ ID NO:31, and SEQ ID NO:32; and at least one of the individual nucleic acids is selected from the group consisting of SEQ ID NO:1, SEQ ID NO:2, SEQ ID NO:5, SEQ ID NO:6, SEQ ID NO:18, SEQ ID NO:19, SEQ ID NO:22, SEQ ID NO:23, and SEQ ID NO:30. In another embodiment, at least four individual nucleic acids are SEQ ID NO:2, SEQ ID NO:15, SEQ ID NO:18, and SEQ ID NO:22.

In one embodiment of the invention, a relative change in expression as compared to the control sample of at least one nucleic acid selected from the group consisting of SEQ ID NO:1, SEQ ID NO:10, SEQ ID NO:12, SEQ ID NO:14, SEQ ID NO:15, SEQ ID NO:19, SEQ ID NO:22, SEQ ID NO:25, SEQ ID NO:30, and SEQ ID NO:33 is characteristic of the beta-IFN therapy response.

In one embodiment of the invention, relative decreased expression as compared to the control sample of at least one nucleic acid selected from the group consisting of SEQ ID NO:1, SEQ ID NO:10, SEQ ID NO:12, SEQ ID NO:25, and SEQ ID NO:30 is characteristic of the beta-IFN therapy response. In a specific embodiment, the relative decrease is at least about 1.5-fold.

In one embodiment of the invention, relative increased expression as compared to the control sample of at least one nucleic acid selected from the group consisting of SEQ ID NO:14, SEQ ID NO:15, SEQ ID NO:19, SEQ ID NO:22, and SEQ ID NO:33 is characteristic of the beta-IFN therapy response. In a specific embodiment, the relative increase is at least about 1.5-fold.

An embodiment of the invention is method of predicting treatment response of a multiple sclerosis patient to beta-IFN therapy comprising the steps of: obtaining a sample of peripheral blood mononuclear cells from the patient; contacting the ample of peripheral blood mononuclear cells with a therapeutically effective amount of beta-IFN; isolating RNA from the sample; determining the relative expression profile in the isolated RNA of at least four individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and comparing the relative expression profile of the individual nucleic acids to a control sample, wherein the beta-IFN is predicted to be therapeutically effective if the relative expression profile is characteristic of a beta-IFN therapy response.

An embodiment of the invention is a method of screening a multiple sclerosis patient for the presence of neutralizing antibody to beta-IFN comprising the steps of: obtaining a sample of peripheral blood mononuclear cells from the patient; isolating RNA from the sample; determining the relative expression profile in the isolated RNA of at least four individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and comparing the relative expression profile of the individual nucleic acids to a control sample, wherein neutralizing antibody to beta-IFN is present if the relative expression profile is characteristic of a blocked beta-IFN therapy response.

An embodiment of the invention is a method of monitoring a multiple sclerosis patient taking beta-IFN comprising the steps of: obtaining a sample of peripheral blood mononuclear cells from the patient; isolating RNA from the sample; determining the relative expression profile in the isolated RNA of at least two individual nucleic acids, wherein at least one individual nucleic acid is selected from the group consisting of SEQ ID NO:15, SEQ ID No: 29, SEQ ID NO:31, and SEQ ID NO:32, and at least one individual nucleic acid is selected from the group consisting of SEQ ID NO:1, SEQ ID NO:2, SEQ ID NO:5, SEQ ID NO:6, SEQ ID NO:18, SEQ ID NO:19, SEQ ID NO:22, SEQ ID NO:23, and SEQ ID NO:30; and comparing the relative expression profile of the individual nucleic acids to a control sample, wherein the beta-IFN is predicted to be therapeutically effective if the relative expression profile is characteristic of a beta-IFN therapy response.

An embodiment of the invention is a method of monitoring a multiple sclerosis patient taking glatiramer acetate comprising the steps of: obtaining a sample of peripheral blood mononuclear cells from the patient; isolating RNA from the sample; determining the relative expression profile in the isolated RNA of at least three individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and comparing the relative expression profile of the individual nucleic acids to a control sample, wherein the glatiramer acetate is therapeutically effective if the relative expression profile is characteristic of a glatiramer acetate therapy response.

In one embodiment, a relative change in expression as compared to a control sample of at least one nucleic acid selected from the group consisting of SEQ ID NO:7, SEQ ID NO:13, SEQ ID NO:16, SEQ ID NO:22, SEQ ID NO:27, and SEQ ID NO:34 is characteristic of the glatiramer acetate therapy response.

In another embodiment, relative decreased expression as compared to a control sample of at least one nucleic acid selected from the group consisting of SEQ ID NO:7, SEQ ID NO:13, SEQ ID NO:16, SEQ ID NO:27, and SEQ ID NO:34 is characteristic of the glatiramer acetate therapy response. In a specific embodiment, the relative decrease is at least about 1.5-fold.

In another embodiment, relative increased expression as compared to a control sample of SEQ ID NO:22 is characteristic of the glatiramer acetate therapy response. In a specific embodiment, the relative increase is at least about 1.5-fold.

In one embodiment, the at least three individual nucleic acids are SEQ ID NO:1, SEQ ID NO:2, and SEQ ID NO:18.

An embodiment of the invention is a method of predicting treatment response of a multiple sclerosis patient to glatiramer acetate therapy comprising the steps of: obtaining a sample of peripheral blood mononuclear cells from the patient; contacting the sample of peripheral blood mononuclear cells with a therapeutically effective amount of glatiramer acetate; isolating RNA from the sample; determining the relative expression profile in the isolated RNA of at least three individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and comparing the relative expression profile of the individual nucleic acids to a control sample, wherein the glatiramer acetate is predicted to be therapeutically effective if the relative expression profile is characteristic of a glatiramer acetate therapy response.

An embodiment of the inention is an array comprising nucleic acid probes attached to a solid surface, wherein the nucleic acid probes are complementary to at least five of the nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34.

In a specific embodiment, the solid surface is nitrocellulose or glass.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a schematic representation of the gene expression profiling procedure;

FIG. 2 shows gene expression profiling of PBMC in response to in vitro treatment with beta-IFN or GA.;

FIG. 3 shows the expression of selected genes analyzed by real-time PCR;

FIG. 4 shows the blocking effect of serum beta-IFN antibody on the immunomodulatory properties of beta-IFN;

FIG. 5 shows the detection of blocking effect of a panel of serum beta-IFN antibodies by gene expression profiling;

FIG. 6 representative hybridization results of self-paired ex vivo analysis before and after beta-IFN or GA treatment; and

FIGS. 7A-C are an ex vivo analysis of PBMC by gene expression profiling for the treatment effect of beta-IFN or GA compared to a control in MS patients.

### DETAILED DESCRIPTION OF THE INVENTION

I. Definitions

As used herein, the use of the word “a” or “an” when used in conjunction with the term “comprising” in the sentences and/or the specification may mean “one,” but it is also consistent with the meaning of “one or more,” “at least one,” and “one or more than one.” As used herein “another” may mean at least a second or more. Still further, the terms “having”, “including”, “containing” and “comprising” are interchangeable and one of skill in the art is cognizant that these terms are open ended terms.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all. Depending on the analysis, one skilled in the art knows which background signal calculation to use.

As used herein, the expressions “cell”, “cell line”, and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

As used herein, a “control sample” refers to any patient sample or isolated RNA sample that serves as a reference in the present invention. In certain embodiments of the invention, the control sample is isolated from patients without MS. In other embodiments of the invention, the control sample is negative for beta-IFN neutralizing antibody. In other embodiments of the invention, the control sample is from a patient who has not been treated with beta-IFN. In other embodiments of the invention, the control sample is from a patient who has not been treated with GA. In one embodiment, the control sample can be a baseline sample taken from a patient before beginning a drug treatment regimen, the drugs being either beta-IFN or GA. Alternatively, the control sample can be obtained from an isolated supply. The control sample may be from an individual or from a population pool that are known to not have been treated with the drug of interest, for example beta-IFN or GA.

As used herein, an “expression profile” or “gene expression profile” comprises measurement of a plurality of mRNAs to indicate the relative expression or relative abundance of any particular transcript. The compilation of the expression levels of all of the mRNA transcripts sampled at any given time point in any given sample comprises the gene expression profile. Within eukaryotic cells, there are hundreds to thousands of signaling pathways that are interconnected. For this reason, changes in the levels or activity of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “characteristic expression profile” of particular transcript alterations which are related to the disruption of function. For example, in certain embodiments of the inventions, the characteristic expression profile is in response to a gene expression disruption caused by beta-IFN treatment of a patient, and the expression profile is referred to as “characteristic of a beta-IFN therapy response.” In certain embodiments of the inventions, the characteristic expression profile is in response to GA treatment of a patient, and the expression profile is referred to as “characteristic of a GA therapy response.”

The term “hybridizing specifically to”, refers to the binding, duplexing, or hybridizing of a molecule only to a particular nucleotide sequence under stringent conditions when that sequence is present in a complex mixture (e.g., total cellular) DNA or RNA. The term “stringent conditions” refers to conditions under which a probe will hybridize to its target subsequence, but to no other sequences. Stringent conditions are sequence-dependent and will be different in different circumstances. One skilled in the art knows how to select such conditions. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is at least about 0.01 to 1.0 M Na ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The term “mismatch control” refers to a probe that has a sequence deliberately selected not to be perfectly complementary to a particular target sequence. The mismatch control typically has a corresponding test probe that is perfectly complementary to the same particular target sequence. The mismatch may comprise one or more bases. While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “mRNA” refers to transcripts of a gene. Transcripts are RNA including, for example, mature messenger RNA ready for translation, products of various stages of transcript processing. Transcript processing may include splicing and degradation.

The terms “nucleic acid” or “nucleic acid molecule” refer to a deoxyribonucleotide or ribonucleotide polymer in either single-or double-stranded form, and unless otherwise limited, would encompass known analogs of natural nucleotides that can function in a similar manner as naturally occurring nucleotides.

An “oligonucleotide” is a single-stranded nucleic acid at least 2 bases in length.

The term “overexpression” means that the relative expression for a particular gene is higher in one sample as compared to another sample. Parameters for overexpression may change as necessary for a particular algorithm. For example, it is contemplated that a gene may not be considered overexpressed unless its expression is at least 1.2, 1.5, 2, or 3 times higher than the control sample.

The term “polypeptide” as used herein is used interchangeably with the term “protein” and is defined as a molecule which comprises more than one amino acid subunit. The polypeptide may be an entire protein or it may be a fragment of a protein, such as a peptide or an oligopeptide. The polypeptide may also comprise alterations to the amino acid subunits, such as methylation or acetylation.

As used herein a “probe” is defined as an oligonucleotide capable of binding to a target nucleic acid of complementary sequence through one or more types of chemical bonds, usually through complementary base pairing, usually through hydrogen bond formation. As used herein, an oligonucleotide probe may include natural (ie. A, G, C, or T) or modified bases (7-deazaguanosine, inosine, etc.). In addition, one skilled in the art recognizes that the bases in oligonucleotide probe may be joined by a linkage other than a phosphodiester bond, so long as it does not interfere with hybridization. Thus, oligonucleotide probes may be peptide nucleic acids in which the constituent bases are joined by peptide bonds rather than phosphodiester linkages.

The term “quantifying” when used in the context of quantifying transcription levels of a gene can refer to absolute or to relative quantification. Absolute quantification may be accomplished by inclusion of known concentration(s) of one or more target nucleic acids (e.g. control nucleic acids such as Bio B or with known amounts of the target nucleic acids themselves) and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

As used herein, the term “relative gene expression” or “relative expression” in reference to a gene refers to the relative abundance of the same gene expression product, usually an mRNA, in different cells or tissue types. In a preferred embodiment, the expression of a gene in a MS sample is compared to MS samples from the same patient taken at different time points, or it is compared to MS samples from different patients. In a specific embodiment, the MS sample is compared to samples taken from the same patient at time points after drug treatment. In a preferred embodiment, the drug treatment is P—IFN therapy or GA therapy. In another preferred embodiment, the MS sample is compared to a sample of a normal patient who does not have MS.

The term “sample” as used herein indicates a patient sample containing at least one cell. Appropriate samples for the present invention include peripheral blood mononuclear cells (PBMCs). One with skill in the art realizes that PBMCs are cells in the bloodstream that have one round nucleus. Such cells comprise lymphocytes and monocytes.

“Subsequence” refers to a sequence of nucleic acids that comprise a part of a longer sequence of nucleic acids.

The term “target nucleic acid” refers to a nucleic acid (often derived from a biological sample), to which the oligonucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid may refer to the specific subsequence of a larger nucleic acid to which the probe is directed or to the overall sequence (e.g., gene or mRNA) whose expression level it is desired to detect. The difference in usage will be apparent from context.

II. Gene Expression Analysis

The present invention measures genes selected for their demonstrated association with MS activity and high susceptibility to beta-IFN or GA treatment (see Table 1). The selected genes include IFN inducible genes to detect an early treatment response and other genes encoding relevant inflammatory biomarkers regulated by beta-IFN or GA based on published evidence. In one embodiment, the selected genes are spotted on a low-cost nylon membrane matrix for easy hybridization and quantification. Ready-to-use membranes pre-spotted with cDNA require one-step hybridization with labeled sample cDNA. The results are analyzed quantitatively by a commonly available software. The whole process takes 8-12 hours and can be performed in any routine clinical laboratory at roughly 1/1,000 cost of conventional cDNA microarray analysis.

In general, gene expression data may be gathered in any way that is available to one of skill in the art. Although many methods provided herein are powerful tools for the analysis of data obtained by highly parallel data collection systems, many such methods are equally useful for the analysis of data gathered by more traditional methods. Commonly, gene expression data is obtained by employing an array of probes that hybridize to several, and even thousands or more different transcripts. Such arrays are often classified as microarrays or macroarrays, and this classification depends on the size of each position on the array.

In one embodiment, the present invention also provides a method wherein nucleic acid probes are immobilized on or in a solid or semisolid support in an organized array. Oligonucleotides can be bound to a support by a variety of processes, including lithography, and where the support is solid, it is common in the art to refer to such an array as a “chip”, although this parlance is not intended to indicate that the support is silicon or has any useful conductive properties.

One embodiment of the invention involves monitoring gene expression by (1) providing a pool of target nucleic acids comprising RNA transcript(s) of one or more target gene(s), or nucleic acids derived from the RNA transcript(s); (2) hybridizing the nucleic acid sample to a array of probes (including control probes); and (3) detecting the hybridized nucleic acids and calculating a relative expression (transcription) level. A. Providing a Nucleic Acid Sample.

One of skill in the art will appreciate that in order to measure the transcription level (and thereby the expression level) of a gene or genes, it is desirable to provide a nucleic acid sample comprising mRNA transcript(s) of the gene or genes, or nucleic acids derived from the mRNA transcript(s). As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

In a particularly preferred embodiment, where it is desired to quantify the transcription level (and thereby expression) of a one or more genes in a sample, the nucleic acid sample is one in which the concentration of the mRNA transcript(s) of the gene or genes, or the concentration of the nucleic acids derived from the mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes. Where more precise quantification is required appropriate controls can be run to correct for variations introduced in sample preparation and hybridization as described herein. In addition, serial dilutions of “standard” target mRNAs can be used to prepare calibration curves according to methods well known to those of skill in the art. Of course, where simple detection of the presence or absence of a transcript is desired, no elaborate control or calibration is required.

In the simplest embodiment, such a nucleic acid sample is the total mRNA isolated from a biological sample. The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

The nucleic acid (either genomic DNA or mRNA) may be isolated from the sample according to any of a number of methods well known to those of skill in the art. One of skill will appreciate that where alterations in the copy number of a gene are to be detected genomic DNA is preferably isolated. Conversely, where expression levels of a gene or genes are to be detected, preferably RNA (mRNA) is isolated.

Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization with Nucleic Acid Probes, Part I. Theory and Nucleic Acid Preparation, P. Tijssen, ed. Elsevier, N.Y. (1993)).

In a preferred embodiment, the total nucleic acid is isolated from a given sample using, for example, an acid guanidinium-phenol-chloroform extraction method and polyA mRNA is isolated by oligo dT column chromatography or by using (dT)ₙ magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The array may then include probes specific to the internal standard for quantification of the amplified nucleic acid.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

In a particularly preferred embodiment, the sample mRNA is reverse transcribed with a reverse transcriptase and a primer consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook, supra.) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 provide a protocol that uses two rounds of amplification via in vitro transcription to achieve greater than 106 fold amplification of the original starting material thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lamda vectors designed for Cre-loxP plasmid subcloning (see e.g., Palazzolo et al., Gene, 88: 25-36 (1990)).

In a particularly preferred embodiment, a high activity RNA polymerase (e.g. about 2500 units/μL for T7, available from Epicentre Technologies) is used. B. Labeling nucleic acids.

In a preferred embodiment, the hybridized nucleic acids are detected by detecting one or more labels attached to the sample nucleic acids. The labels may be incorporated by any of a number of means well known to those of skill in the art. However, in a preferred embodiment, the label is simultaneously incorporated during the amplification step in the preparation of the sample nucleic acids. Thus, for example, polymerase chain reaction (PCR) with labeled primers or labeled nucleotides will provide a labeled amplification product. In a preferred embodiment, transcription amplification, as described above, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., .sup.3H,.sup.125 I, .sup.35 S, .sup.14 C, or .sup.32 P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are preferred and easily added during an in vitro transcription reaction. In a preferred embodiment, fluorescein labeled UTP and CTP are incorporated into the RNA produced in an in vitro transcription reaction as described above.

C. Modifying Sample to Improve Signal/Noise Ratio.

The nucleic acid sample may be modified prior to hybridization to the high density probe array in order to reduce sample complexity thereby decreasing background signal and improving sensitivity of the measurement. In one embodiment, complexity reduction is achieved by selective degradation of background mRNA. This is accomplished by hybridizing the sample mRNA (e.g., polyA RNA) with a pool of DNA oligonucleotides that hybridize specifically with the regions to which the probes in the array specifically hybridize. In a preferred embodiment, the pool of oligonucleotides consists of the same probe oligonucleotides as found on the array.

The pool of oligonucleotides hybridizes to the sample mRNA forming a number of double stranded (hybrid duplex) nucleic acids. The hybridized sample is then treated with RNase A, a nuclease that specifically digests single stranded RNA. The RNase A is then inhibited, using a protease and/or commercially available RNase inhibitors, and the double stranded nucleic acids are then separated from the digested single stranded RNA. This separation may be accomplished in a number of ways well known to those of skill in the art including, but not limited to, electrophoresis and gradient centrifugation. However, in a preferred embodiment, the pool of DNA oligonucleotides is provided attached to beads forming thereby a nucleic acid affinity column. After digestion with the RNase A, the hybridized DNA is removed simply by denaturing (e.g., by adding heat or increasing salt) the hybrid duplexes and washing the previously hybridized mRNA off in an elution buffer.

The undigested mRNA fragments which will be hybridized to the probes in the array are then preferably end-labeled with a fluorophore attached to an RNA linker using an RNA ligase. This procedure produces a labeled sample RNA pool in which the nucleic acids that do not correspond to probes in the array are eliminated and thus unavailable to contribute to a background signal.

Another method of reducing sample complexity involves hybridizing the mRNA with deoxyoligonucleotides that hybridize to regions that border on either side of the regions to which the array probes are directed. Treatment with RNAse H selectively digests the double stranded (hybrid duplexes) leaving a pool of single-stranded mRNA corresponding to the short regions (e.g., 20 mer) that were formerly bounded by the deoxyolignucleotide probes and which correspond to the targets of the array probes and longer mRNA sequences that correspond to regions between the targets of the probes of the array. The short RNA fragments are then separated from the long fragments (e.g., by electrophoresis), labeled if necessary as described above, and then are ready for hybridization with the high density probe array.

In a third approach, sample complexity reduction involves the selective removal of particular (preselected) mRNA messages. In particular, highly expressed mRNA messages that are not specifically probed by the probes in the array are preferably removed. This approach involves hybridizing the polyA mRNA with an oligonucleotide probe that specifically hybridizes to the preselected message close to the 3′ (poly A) end. The probe may be selected to provide high specificity and low cross reactivity. Treatment of the hybridized message/probe complex with RNase H digests the double stranded region effectively removing the polyA tail from the rest of the message. The sample is then treated with methods that specifically retain or amplify polyA RNA (e.g., an oligo dT column or (dT)n magnetic beads). Such methods will not retain or amplify the selected message(s) as they are no longer associated with a polyA.sup.+tail. These highly expressed messages are effectively removed from the sample providing a sample that has reduced background mRNA.

III. Hybridization Array Design

A. Probe Composition

One of skill in the art will appreciate that an enormous number of array designs are suitable for the practice of this invention. The array will typically include a number of probes that specifically hybridize to the nucleic acid expression which is to be detected. In a preferred embodiment, the array will include one or more control probes.

1) Test Probes

In its simplest embodiment, the array includes “test probes”. These are oligonucleotides that range from about 5 to about 50 nucleotides, more preferably from about 10 to about 40 nucleotides and most preferably from about 15 to about 40 nucleotides in length. These oligonucleotide probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the array can contain a number of control probes. The control probes fall into three categories referred to herein as a) Normalization controls; b) Expression level controls; and c) Mismatch controls.

a) Normalization Controls.

Normalization controls are oligonucleotide probes that are perfectly complementary to labeled reference oligonucleotides that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few normalization probes are used and they are selected such that they hybridize well (i.e. no secondary structure) and do not match any target-specific probes.

Normalization probes can be localized at any position in the array or at multiple positions throughout the array to control for spatial variation in hybridization efficiently. In a preferred embodiment, the normalization controls are located at the corners or edges of the array as well as in the middle.
b) Expression Level Controls.

Expression level controls are probes that hybridize specifically with constitutively expressed genes in the biological sample. Expression level controls are designed to control for the overall health and metabolic activity of a cell. Examination of the covariance of an expression level control with the expression level of the target nucleic acid indicates whether measured changes or variations in expression level of a gene is due to changes in transcription rate of that gene or to general variations in health of the cell. Thus, for example, when a cell is in poor health or lacking a critical metabolite the expression levels of both an active target gene and a constitutively expressed gene are expected to decrease. The converse is also true. Thus where the expression levels of both an expression level control and the target gene appear to both decrease or to both increase, the change may be attributed to changes in the metabolic activity of the cell as a whole, not to differential expression of the target gene in question. Conversely, where the expression levels of the target gene and the expression level control do not covary, the variation in the expression level of the target gene is attributed to differences in regulation of that gene and not to overall variations in the metabolic activity of the cell.

Virtually any constitutively expressed gene provides a suitable target for expression level controls. Typically expression level control probes have sequences complementary to subsequences of constitutively expressed “housekeeping genes” including, but not limited to the β-actin gene, the transferrin receptor gene, the GAPDH gene, and the like.

c) Mismatch Controls.

Mismatch controls may also be provided for the probes to the target genes, for expression level controls or for normalization controls. Mismatch controls are oligonucleotide probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g. stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross-hybridization to a nucleic acid in the sample other than the target to which the probe is directed. Mismatch probes thus indicate whether a hybridization is specific or not. For example, if the target is present the perfect match probes should be consistently brighter than the mismatch probes. In addition, if all central mismatches are present, the mismatch probes can be used to detect a mutation. Finally, it was also a discovery of the present invention that the difference in intensity between the perfect match and the mismatch probe (I(PM)-I(MM)) provides a good measure of the concentration of the hybridized material.

2) Sample Preparation/Amplification Controls

The array may also include sample preparation/amplification control probes. These are probes that are complementary to subsequences of control genes selected because they do not normally occur in the nucleic acids of the particular biological sample being assayed. Suitable sample preparation/amplification control probes include, for example, probes to bacterial genes (e.g., Bio B) where the sample in question is a biological from a eukaryote.

The RNA sample is then spiked with a known amount of the nucleic acid to which the sample preparation/amplification control probe is directed before processing. Quantification of the hybridization of the sample preparation/amplification control probe then provides a measure of alteration in the abundance of the nucleic acids caused by processing steps (e.g. PCR, reverse transcription, in vitro transcription, etc.).

B. “Test Probe” Selection and Optimization.

In a preferred embodiment, oligonucleotide probes in the array are selected to bind specifically to the nucleic acid target to which they are directed with minimal non-specific binding or cross-hybridization under the particular hybridization conditions utilized.

There, however, may exist 20 mer subsequences that are not unique to a particular mRNA. Probes directed to these subsequences are expected to cross hybridize with occurrences of their complementary sequence in other regions of the sample genome. Similarly, other probes simply may not hybridize effectively under the hybridization conditions (e.g., due to secondary structure, or interactions with the substrate or other probes). Thus, in a preferred embodiment, the probes that show such poor specificity or hybridization efficiency are identified and may not be included either in the array itself (e.g., during fabrication of the array) or in the post-hybridization data analysis.

Thus, in one embodiment, this invention provides for a method of optimizing a probe set for detection of a particular gene. Generally, this method involves providing a array containing a multiplicity of probes of one or more particular length(s) that are complementary to subsequences of the mRNA transcribed by the target gene. In one embodiment the array may contain every probe of a particular length that is complementary to a particular mRNA. The probes of the array are then hybridized with their target nucleic acid alone and then hybridized with a high complexity, high concentration nucleic acid sample that does not contain the targets complementary to the probes. Thus, for example, where the target nucleic acid is an RNA, the probes are first hybridized with their target nucleic acid alone and then hybridized with RNA made from a cDNA library (e.g., reverse transcribed polyA mRNA) where the sense of the hybridized RNA is opposite that of the target nucleic acid (to insure that the high complexity sample does not contain targets for the probes). Those probes that show a strong hybridization signal with their target and little or no cross-hybridization with the high complexity sample are preferred probes for use in the arrays of this invention.

The array may additionally contain mismatch controls for each of the probes to be tested. In a preferred embodiment, the mismatch controls contain a central mismatch. Where both the mismatch control and the target probe show high levels of hybridization (e.g., the hybridization to the mismatch is nearly equal to or greater than the hybridization to the corresponding test probe), the test probe is preferably not used in the array.

In a particularly preferred embodiment, an array is provided containing a multiplicity of oligonucleotide probes complementary to subsequences of the target nucleic acid. The oligonucleotide probes may be of a single length or may span a variety of lengths ranging from 5 to 50 nucleotides. The array may contain every probe of a particular length that is complementary to a particular mRNA or may contain probes selected from various regions of particular mRNAs. For each target-specific probe the array also contains a mismatch control probe; preferably a central mismatch control probe.

The oligonucleotide array is hybridized to a sample containing target nucleic acids having subsequences complementary to the oligonucleotide probes and the difference in hybridization intensity between each probe and its mismatch control is determined. Only those probes where the difference between the probe and its mismatch control exceeds a threshold hybridization intensity (e.g. preferably greater than 10% of the background signal intensity, more preferably greater than 20% of the background signal intensity and most preferably greater than 50% of the background signal intensity) are selected. Thus, only probes that show a strong signal compared to their mismatch control are selected.

The probe optimization procedure can optionally include a second round of selection. In this selection, the oligonucleotide probe array is hybridized with a nucleic acid sample that is not expected to contain sequences complementary to the probes. Thus, for example, where the probes are complementary to the RNA sense strand a sample of antisense RNA is provided. Of course, other samples could be provided such as samples from organisms or cell lines known to be lacking a particular gene, or known for not expressing a particular gene.

Only those probes where both the probe and its mismatch control show hybridization intensities below a threshold value (e.g. less than about 5 times the background signal intensity, preferably equal to or less than about 2 times the background signal intensity, more preferably equal to or less than about 1 times the background signal intensity, and most preferably equal or less than about half background signal intensity) are selected. In this way probes that show minimal non-specific binding are selected. Finally, in a preferred embodiment, the n probes (where n is the number of probes desired for each target gene) that pass both selection criteria and have the highest hybridization intensity for each target gene are selected for incorporation into the array, or where already present in the array, for subsequent data analysis. Of course, one of skill in the art, will appreciate that either selection criterion could be used alone for selection of probes.

One set of hybridization rules for 20 mer probes in this manner is the following: a) Number of As is less than 9; b) Number of Ts is less than 10 and greater than 0; c) Maximum run of As, Gs, or Ts is less than 4 bases in a row; d) Maximum run of any 2 bases is less than 11 bases; e) Palindrome score is less than 6; f) Clumping score is less than 6; g) Number of As+Number of Ts is less than 14; h) Number of As+number of Gs is less than 15. With respect to rule d, requiring the maximum run of any two bases to be less than 11 bases guarantees that at least three different bases occur within any 12 consecutive nucleotide. A palindrome score is the maximum number of complementary bases if the oligonucleotide is folded over at a point that maximizes self complementarity. Thus, for example a 20 mer that is perfectly self-complementary would have a palindrome score of 10. A clumping score is the maximum number of three-mers of identical bases in a given sequence. Thus, for example, a run of 5 identical bases will produce a clumping score of 3 (bases 1-3, bases 2-4, and bases 3-5). If any probe fails one of these criteria (a-h), the probe is not a member of the subset of probes placed on the chip. For example, if a hypothetical probe was 5′-AGCTTTTTTCATGCATCTAT-3′ the probe would not be synthesized on the chip because it has a run of four or more bases (i.e., a run of six). The cross hybridization rules developed for 20 mers were as follows: a) Number of Cs is less than 8; b) Number of Cs in any window of 8 bases is less than 4. Thus, if any probe fails any of either the hybridization ruses (a-h) or the cross-hybridization rules (a-b), the probe is not a member of the subset of probes placed on the chip. These rules eliminate many of the probes that cross hybridize strongly or exhibit low hybridization.

C. Attaching Nucleic Acids to the Solid Surface

The nucleic acid or analogue are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995 (Quantitative monitoring of gene expression patterns with a complementary DNA microarray, Science 270:467-470). This method is especially useful for preparing microarrays of cDNA. See also DeRisi et al., 1996 (Use of a cDNA microarray to analyze gene expression patterns in human cancer, Nature Genetics 14:457-460; Shalon et al., 1996, A DNA microarray system for analyzing complex DNA samples using two-color fluorescent probe hybridization, Genome Res. 6:639-645; and Schena et al., 1995, Parallel human genome analysis; microarray-based expression of 1000 genes, Proc. Natl. Acad. Sci. USA 93:10614-10619). Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Light-directed spatially addressable parallel chemical synthesis, Science 251:767-773; Pease et al., 1994, Light-directed oligonucleotide arrays for rapid DNA sequence analysis, Proc. Natl. Acad. Sci. USA 91:5022-5026; Lockhart et al., 1996, Expression monitoring by hybridization to high-density oligonucleotide arrays, Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al., 1996, High-Density Oligonucleotide arrays, Biosensors & Bioelectronics 11: 687-90). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs. Another preferred method of making microarrays is by use of an inkjet printing process to synthesize oligonucleotides directly on a solid phase.

Other methods for making microarrays; e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller.

IV. Microarray Data Analysis

Although microarray analysis determines the expression levels of thousands of genes in an RNA sample, only a few of these genes will be differentially expressed upon introduction of a particular variable. In the case of the present invention, breast tissues are either docetaxel sensitive or resistant. The identification of the genes which are necessary for classification in order to predict a clinical outcome is an object of the present invention.

For many applications of the present invention, it is desirable to find basis gene sets that are co-regulated over a wide variety of conditions. This allows the method of invention to work well for a large class of profiles whose expected properties are not well circumscribed. A preferred embodiment for identifying such basis gene sets involves clustering algorithms, which are well known to one with skill in the art. (for reviews of clustering algorithms, see, e.g., Fukunaga, 1990, Statistical Pattern Recognition, 2nd Ed., Academic Press, San Diego; Everitt, 1974, Cluster Analysis, London: Heinemann Educ. Books; Hartigan, 1975, Clustering Algorithms, New York: Wiley; Sneath and Sokal, 1973, Numerical Taxonomy, Freeman; Anderberg, 1973, Cluster Analysis for Applications, Academic Press: New York).

In order to obtain basis genesets that contain genes which co-vary over a wide variety of conditions, a plurality of genes are analyzed. In a preferred embodiment, at least 10 or more, preferably at least 50 genes are analyzed. On other embodiments, at least 91 genes are analyzed. Cluster analysis operates on a table of data which has the dimension m×k wherein m is the total number of groups that cluster (in the present invention, two groups are contemplated, docetaxel resistant and docetaxel sensitive) and k is the number of genes measured.

A number of clustering algorithms are useful for clustering analysis. Clustering algorithms use dissimilarities or distances between objects when forming clusters. In some embodiments, the distance used is Euclidean distance, which is known to one with skill in the art, in multidimensional space where I(x,y) is the distance between gene X and gene Y; Xᵢ and Yᵢ are gene expression response under perturbation i. The Euclidean distance may be squared to place progressively greater weight on objects that are further apart. Alternatively, the distance measure may be the Manhattan distance, which is known to a skilled artisan, e.g., between gene X and Y. Again, Xᵢ and Yᵢ are gene expression responses under perturbation i. Some other definitions of distances are Chebychev distance, power distance, and percent disagreement. Another useful distance definition, which is particularly useful in the context of cellular response, is I=1−r, where r is the correlation coefficient between the response vectors X, Y, also called the normalized dot product XY/|X∥Y|.

Various cluster linkage rules are useful for the methods of the invention. Single linkage, a nearest neighbor method, determines the distance between the two closest objects. By contrast, complete linkage methods determine distance by the greatest distance between any two objects in the different clusters. This method is particularly useful in cases when genes or other cellular constituents form naturally distinct “clumps.” Alternatively, the unweighted pair-group average defines distance as the average distance between all pairs of objects in two different clusters. This method is also very useful for clustering genes or other cellular constituents to form naturally distinct “clumps.” Finally, the weighted pair-group average method may also be used. This method is the same as the unweighted pair-group average method except that the size of the respective clusters is used as a weight. This method is particularly useful for embodiments where the cluster size is suspected to be greatly varied (Sneath and Sokal, 1973, Numerical taxonomy, San Francisco. W. H. Freeman & Co.). Other cluster linkage rules, such as the unweighted and weighted pair-group centroid and Ward's method are also useful for some embodiments of the invention. See., e g, Ward, 1963, J. Am. Stat Assn. 58:236, Hartigan, 1975, Clustering algorithms, New York: Wiley.

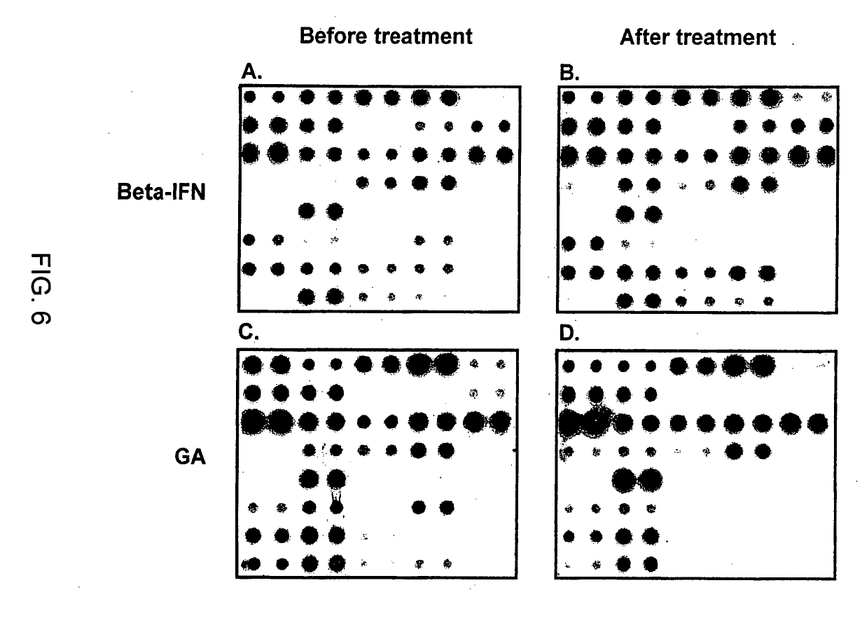

The cluster analysis may be performed using the hclust routine (see, e.g., ‘hclust’routine from the software package S-Plus, MathSoft, Inc., Cambridge, Mass.). Genesets may be defined based on the many smaller branches in the tree, or a small number of larger branches by cutting across the tree at different levels—see the example dashed line in FIG. 6. The choice of cut level may be made to match the number of distinct response pathways expected. If little or no prior information is available about the number of pathways, then the tree should be divided into as many branches as are truly distinct. ‘Truly distinct’ may be defined by a minimum distance value between the individual branches. Preferably, ‘truly distinct’ may be defined with an objective test of statistical significance for each bifurcation in the tree. In one aspect of the invention, the Monte Carlo randomization of the experiment index for each cellular constituent's responses across the set of experiments is used to define an objective test.

Analysis of thousands of data points after performing a microarray experiment in order to identify those key genes which contribute significantly to tissue classification may be accomplished in a variety of ways. One approach may be unsupervised clustering techniques, such as hierarchical clustering, which identifies sets of correlated genes with similar behavior across the experiments, but yields thousands of clusters in a tree-like structure. Self-organizing-maps, or SOM, require a prespecified number and an initial spatial structure of clusters.

In a preferred embodiment of the invention, the microarray data from the breast tissue samples is analyzed by a supervised clustering algorithm. Any number of suitable algorithms may be used. For example, see Dettling et al., 2002. Such algorithms may be user-designed or may be previously packaged in a microarray data analysis software system.

R-SVM is a supported vector machine (SVM)-based method for doing supervised pattern recognition(classification) with microarray gene expression data. The method is useful in classification and for selecting a subset of relevant genes according to their relative contribution in the classification. This process is recursive and the accuracy of the classification can be evaluated either on an independent test data set or by cross validation on the same data set. R-SVM also includes an option for permutation experiments to assess the significance of the performance.

V. Isolation of PBMCs

“Peripheral blood mononuclear cells” (PBMCs) refers to a mixture of monocytes and lymphocytes. One with skill in the art is aware of several methods for isolating PBMCs. In one embodiment of the invention, PBMCs can be isolated from whole blood samples using different density gradient centrifugation procedures. Anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to get rid of some contaminants before cell type and cell viability can be confirmed.

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Patients and Blood Specimens

Thirty patients with either relapsing-remitting MS or secondary progressive MS were studied. The first batch of blood specimens was taken from fifteen patients who had not been treated with beta-IFN or GA for at least 24 months prior to the study. The second batch of blood specimens was taken from thirty patients who had received treatment with either beta-IFN-1a (Avonex® or Rebif®) or GA for 2-8 years. A group of 9 healthy volunteers (6 female and 3 male individuals) were included to provide preparation of peripheral blood mononuclear cells (PBMC) as indicator cells for gene expression profiling by in vitro treatment of the drugs.

### Example 2

### In Vitro Treatment of PBMC with Beta-IFN or GA and Serum Neutralizing Antibody

Culture medium used in the study was RPMI-1640 supplemented with 10% heat-inactivated fetal calf serum and L-glutamine, sodium pyruvate, non-essential amino-acids, and 10 mM HEPES buffer (Hyclone, Logan, Utah). Beta-IFN and GA used in this study were obtained from Berlex (Richmond, Calif.) and Teva pharmaceuticals (Kansas City, Mo.), respectively. PBMC were prepared by Ficoll density gradient centrifugation and resuspended in 5% FCS RPMI 1640. For in vitro treatment with beta-IFN or GA, 10⁷ cells were cultured in the presence of 1.5 ng/ml beta-IFN or 50 μg/ml GA for 6, 12, 18 and 24 hours in a humidified atmosphere of 5% CO₂ at 37° C. It was determined in a series of pilot experiments that a 24-hour incubation time yielded best gene expression profiling results. PBMC cultured under the same conditions in the absence of the drug served as a control. Subsequently, cells were collected for RNA preparation. In experiments to determine the blocking effect of beta-IFN neutralizing antibody, positive serum samples derived from MS patients treated with beta-IFN were added, at 1:100 dilution, to separate PBMC cultures containing beta-IFN (1.5 ng/ml). Serum specimens tested negative for beta-IFN antibody by ELISA and cytopathic assay were used as control sera.

### Example 3

RNA Isolation

Total RNA was isolated from PBMC specimens using the RNeasy kit (Qiagen, Valencia, Calif.) according to the manufacturer's instructions. To obtain high quality RNA, contaminating DNA was efficiently removed from RNA samples by digestion with DNase I (Qiagen, Valencia, Calif.) in the process of RNA isolation. The integrity of isolated RNA was verified on a gel prior to cDNA probe labeling.

### Example 4

Preparation of cDNA Gene Array

Each nylon membrane-based cDNA gene array contained 36 selected genes, including beta actin as a normalizer and pUC18 as a negative control gene. All cDNA probe sequences were confirmed by the BLAST database. Individual cDNA clones were generated for all selected genes and utilized as templates to prepare cDNA probes by RT-PCR. The conditions for cDNA spotting and concentrations were optimized in a series of pilot experiments for best hybridization results. 0.1 μg of cDNA probe of each gene was immobilized on nylon membranes (Amersham Pharmarcia, Piscatway, N.J.) using vacuum blotting (Bio-Rad, Hercules, Calif.) and crosslinked by UV at a dose of 1.2×10⁵ microJoules/cm² using Stratalinker, (Stratagene, La Jolla, Calif.).

### Example 5

### Gene Expression Profiling Procedure

The procedure was comprised of sample cDNA preparation, labeling, hybridization, chemiluminescent detection of hybridized cDNA and densometric quantification. Each gene was hybridized in duplicate. For sample cDNA preparation, labeling and hybridization, 5 μg of RNA was converted into first stand cDNA using random hexamer. Subsequently, 20 pmol of mixed sense primers of 36 selected genes, 1 unit of Taq DNA polymerase, 10 nmol dNTP and 2 nmol of DIG-11-dUTP (Roche, Indianapolis, Ind.) were added into the product of reverse transcription reactions and H₂O was supplemented to bring the total reaction volume up to 50 μl. The reaction was carried out under the following conditions: 94° C. for 30 sec, 20 cycles of 94° C. for 30 sec, 50° C. for 20 sec, 72° C. for 50 sec, with the final extension at 72° C. for 7 min. Free DIG-11-dUTP mixed with labeled cDNA was removed by PCR purification column (Qiagen, Valencia, Calif.) before labeled cDNA was used for hybridization. Prehybridization of cDNA probe-spotted membrane was performed at 45° C. for at least 30 min with a minimum of 0.2 ml hybridization buffer/cm² and freshly denatured labeled cDNA were added into hybridization solution with continued hybridization overnight. The hybridized membranes were rinsed twice with 2×SSC, 0.1% SDS and washed subsequently with 0.1×SSC, 0.1% SDS for 30 min. For chemiluminescent detection of hybridized cDNA, membranes were blocked with 5% blocking reagent (Roche, Indianapolis, Ind.) at room temperature for 30 min and then incubated with 1:10,000 diluted anti-DIG antibody conjugated with alkali phosphatase at room temperature for another 30 min. After washing in 0.1% Tween 20 and 0.1 M maleic acid solution (2 times in 15 min), membranes were incubated with CDPstar alkali phosphatase substrate (Amersham Pharmarcia, Piscatway, N.J.) for 2 min and exposed to X-ray films. To determine signal intensities of hybridized dots, optimally exposed films were scanned by a digital scanning device (Gel Doc 1000, Bio-Rad, Hercules, Calif.). After spot intensities were normalized with beta-actin gene on each individual array, signal intensities of the genes were analyzed using Scanalyze 2.5 software according to the manufacturer's instructions. The differences in the gene expression between the two arrays were analyzed by dividing intensities of gene spots on one array by the corresponding spots on the other array after both arrays were normalized with the beta-actin gene. A net change in gene expression by 150%-200% (moderate significance) or by >200% (high significance) was considered significant.

### Example 6

Quantitative real-time RT-PCR

Quantitative real-time RT-PCR was performed on an ABI Prism 7000 sequence detection system (Applied Biosystems, Foster City, Calif.). Beta-actin was used for sample normalization. PCR primers were designed using Primeb Express software (Applied Biosystems, Foster City, Calif.) according to criteria recommended by the manufacturer. The most efficient primers ranged from 18-24 nucleotides with resulting amplicon ranging in size from 75-100 bp. The amplification protocol used was described as follows: 1 μg of RNA was reverse transcribed into cDNA with random hexamers (Invitrogen, Carlsbad, Calif.), and 1 μl of synthesized cDNA product was subsequently added into PCR reaction mix containing 25 μl of 2 x SybGreen master mix (Applied Biosystems, Foster City, Calif.), 23 μl of H₂0, 1 μl of each 10 μM primer. PCR reaction was programmed as 10 min at 94° C. for denaturing and TaqGold polymerase activation followed by 40 thermalcycles of 20 sec at 94° C., 20 sec at 55° C. and 40 sec at 72° C. Relative quantification of gene expression was calculated using delta CT method based on signal intensity of the PCR reactions according to the following formula: 2^{-ACT=[}2^{-(sample Ct-beta actin Ct)}] (Ct=threshold cycle). All reactions were performed in triplicate and results were confirmed by at least one additional independent run.

### Example 7

### Statistical Analysis

Relative expression of each gene in each group of MS patients was analyzed for their normality using the Shapiro-Wilk test. Differences of the relative expression levels of selected genes between MS groups were calculated using the student's t test for normally distributed variables and the nonparametric Mann-Whitney test for non-normally distributed variables. A p value of less than 0.05 was considered statistically significant.

### Example 8

### Characterization of a Novel Gene Expression Profiling Technology for Evaluation of PBMC Responses to In Vitro Treatment with Beta-IFN or GA

A set of genes encoding interferon-inducible proteins and molecules involved in inflammation, T cell trafficking and apoptotic processes were selected for gene profiling. The selection was based on their relevance to MS or their demonstrated susceptibility to in vivo and in vitro regulation by beta-IFN or GA (Table 1).

TABLE 1
Description of selected genes in an array format

| Array | | | | |
|---|---|---|---|---|
| position | Genes | Description | Genbank Accession | SEQ ID NO |
| (1A) (1B) | TNFα | Tumor necrosis factor alpha | X01394 | 1 |
| (2A) (2B) | IL-10 | Interleukin 10 | M57627 | 2 |
| (3A) (3B) | IL-2R | Interleukin-2 receptor | X01057 | 3 |
| (4A) (4B) | IL-5 | Interleukin 5 | X04688 | 4 |
| (5A) (5B) | IL-2 | Interleukin 2 | U25676 | 5 |
| (6A) (6B) | IL-4 | Interleukin 4 | M13982 | 6 |
| (7A) (7B) | Fas | Fas | M67454 | 7 |
| (8A) (8B) | CXCR4 | Chemokine receptor-4 | AF025375 | 8 |
| (1C) (1D) | TGFβ1 | Transforming growth factor Beta 1 | X02812 | 9 |
| (2D) (2D) | MMP-9 | Matrix metalloproteinase 9 | NM_004994 | 10 |
| (3C) (3D) | IL-6 | Interleukin 6 | M14584 | 11 |
| (4C) (4D) | NF-ₖB | Nuclear factor kappa B | X61499 | 12 |
| (5C) (5D) | CXCR3 | G protein-coupled receptor 9 | NM_001504 | 13 |
| (6C) (6D) | ICAM-1 | Intercellular adhesion molecule-1 | J03132 | 14 |
| (7C) &D) | MxA | Interferon-induced cellular resistance mediator | M30817 | 15 |
| | | protein | | |
| (8C) (8D) | CCR5 | CC chemokine receptor 5 | AF031237 | 16 |
| (1E) (1F) | RANTES | Regulated upon activation, normal T-cell | M21121 | 17 |
| | | expressed and secreted | | |
| (2E) (2F) | IFNγ | Interferon IFN-gamma | X13274 | 18 |
| (3E) (3F) | IL-12R B2 | Interleukin 12 receptor Beta 2 | U64198 | 19 |
| (4E) (4F) | IL-8 | Interleukin 8 | BC013615 | 20 |
| (5E) (5F) | CCR3 | CC chemokine receptor 3 | AF247361 | 21 |
| (6E) (6F) | IL-12 p40 | Interleukin 12 40 kDa subunit | M65272 | 22 |
| (7E) (7F) | IL-13 | Interleukin 13 | AF377331 | 23 |
| (8E) (8F) | IL-15Ra | Interleukin 15 receptor, alpha | NM_002189 | 24 |
| (1G) (1H) | VLA-4 | Intergrin alpha 4 | L12002 | 25 |
| (2G) (2H) | Caspase-3 | Apoptosis-related cysteine protease | XM_054686 | 26 |
| (3G) (3H) | P-selectin | Selectin P | NM_003005 | 27 |
| (4G) (4H) | LFA-1 | Leukocyte-associated molecule-1 alpha subunit | Y00796 | 28 |
| (5G) (5H) | IP-10 | Chemokine (C-X-C motif) ligand 10 | NM_001565 | 29 |
| (6G) (6H) | IL-1b | Interleukin 1 beta | M15330 | 30 |
| (7G) (7H) | 1-8U | Interferon-inducible gene family | X57352 | 31 |
| (8G) (8H) | 1-8D | Interferon-inducible gene family | X57351 | 32 |
| (1l) (1J) | iNos | Inducible nitric oxide synthase | AF049656 | 33 |
| (2l) (2J) | ApoE | Apolipoprotein E | BC003557 | 34 |
| (3l) (3J) | β—Actin | Beta actin | XO00351 | |
| (4l) (4J) | pUC18 fragment | PCR amplicon from bacteria plasmid | | |

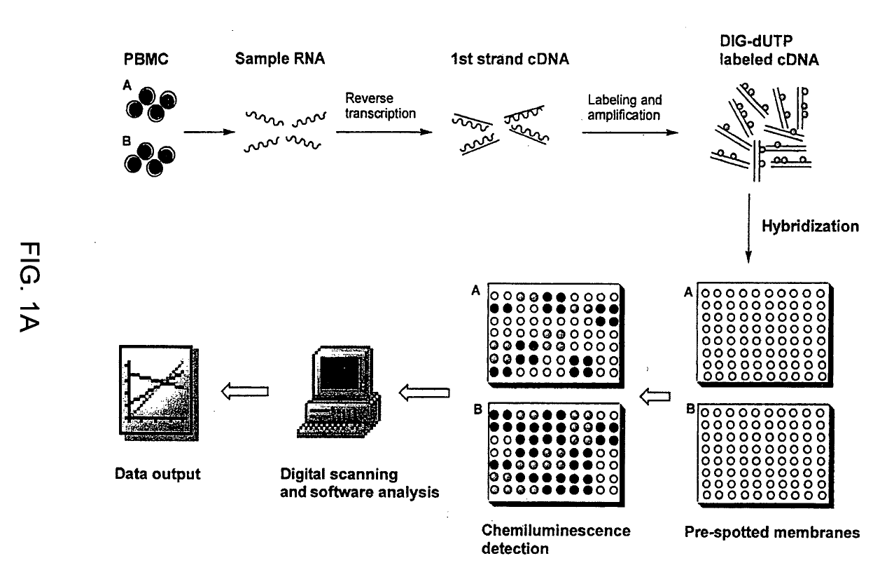

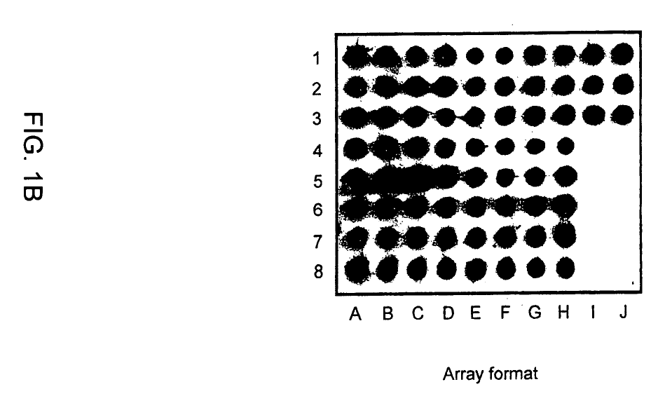

A series of pilot experiments was performed to optimize the experimental conditions for the preparation of the gene array and the profiling procedure. The system required approximately 5 μg sample RNA (5 ml whole blood) for reverse-transcription and labeling for best hybridization results. A total of more than 70 array membranes were prepared in two batches under the same experimental conditions. There were no significant batch differences as determined in quality-control experiments. FIG. 1 shows a schematic representation of the general procedure for gene expression profiling experiments.

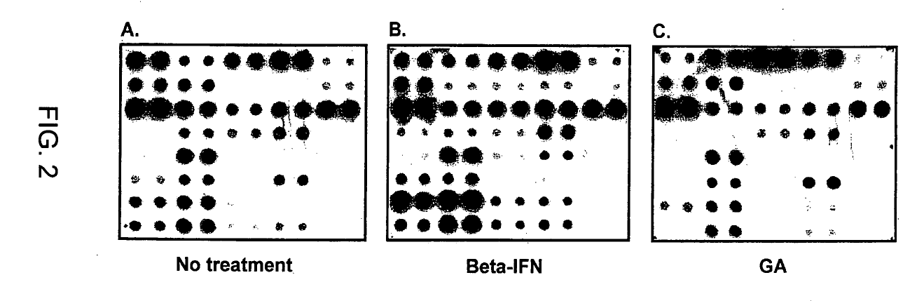

PBMC preparations derived from healthy individuals were treated in culture with beta-IFN (1.5 ng/ml) or GA (50 μg/ml), respectively, and were subsequently analyzed by gene expression profiling to discern specific responses to the drugs. As shown in a representative experiment (FIG. 2), beta-IFN and GA appeared to induce a distinct gene regulation profile characteristic of each drug. Beta-IFN and GA regulated antagonistically the expression of certain genes, including MMP-9, Fas, IL-1b and TNF-alpha, but had a synergistic effect on other genes (e.g. IP-10, CCR5). The complete gene expression profiling of a panel of PBMC preparations in response to beta-IFN or GA is summarized in Table 2a and 2b.

TABLE 2a
Gene expression profiling of PBMC in response to in vitro treatment with Beta-IFN

| Gene clusters | Genes | NS1 | NS2 | NS3 | NS4 | NS5 | MS1 | MS2 | MS3 |
|---|---|---|---|---|---|---|---|---|---|
| IFN inducible genes | IP-10 | ↑ | ↑ | ↑ | ↑ | ↑ | ↑ | ↑ | ↑ |
| | 1-8U | ↑ | ↑ | ↑ | ↑ | ↑ | ↑ | ↑ | ↑ |
| | 1-8D | ↑ | ↑ | ↑ | ↑ | ↑ | ↑ | ↑ | ↑ |
| | MxA | ↑ | ↑ | ↑ | ↑ | ↑ | ↑ | ↑ | ↑ |
| Apoptosis/ | Fas | ↑ | ↑ | — | ↑ | ↑ | ↑ | ↑ | ↑ |
| transcriptional | Caspase-3 | — | — | — | — | ↑ | ↑* | — | ↑ |
| factor | NF-kB | ↓ | — | — | — | ↓ | — | ↓ | — |
| Adhesion | MMP-9 | ↓ | ↓ | ↓ | ↓ | ↓ | ↓ | ↓ | ↓ |
| molecules/cell | LFA-1 | ↓* | ↓ | — | ↓ | ↓* | ↓ | — | ↓ |
| trafficking | ICAM-1 | — | — | — | ↑ | ↑ | — | — | |
| Inflammatory | CXCR3 | ↑ | — | — | ↑ | — | ↑ | ↑ | ↑ |
| cytokines/ | 1L-14Ra | ↑ | ↑ | ↑ | ↑ | ↑ | ↑ | ↑ | ↑* |
| chemokines | CCR5 | ↑ | ↑ | — | ↑ | ↑ | — | ↑ | ↑ |
| and receptors | CCR3 | ↑ | ↑ | ↑ | ↑ | ↑ | ↑ | ↑ | ↑ |
| | TNFa | — | — | — | — | ↓ | ↓ | ↓* | — |
| | 1L-1b | ↓ | ↓ | ↓ | — | ↓ | ↓ | ↓ | ↓ |
| | 1L-8 | ↓ | ↓ | ↓ | — | ↓ | ↓ | ↓ | ↓ |

PBMC derived from five healthy individuals (NS) and three untreated MS patients (MS) were analyzed by gene expression profiling.
Arrows represent specific changes in gene expression (Beta-lFN treatment/control) by at least two times.
Arrows with asterisks indicate a moderate change in gene expression (>1.5 times and <2 times).

TABLE 2b
Gene expression profiling of PBMC in response to in vitro treatment with GA

| Gene clusters | Genes | NS3 | NS4 | NS5 | NS6 | NS7 | NS8 | NS9 | MS1 | MS2 |
|---|---|---|---|---|---|---|---|---|---|---|
| IFN inducible | IP-10 | ↑ | ↑ | ↑ | ↑ | ↑ | ↑ | ↑ | ↑ | — |
| genes | 1-8U | ↑ | ↑ | — | — | — | ↑ | ↑ | ↑ | ↑ |
| | 1-8D | ↑ | ↑ | — | — | — | ↑ | ↑ | ↑ | ↑ |
| Apoptosis/transcriptional | Fas | ↓ | ↓ | ↓ | ↓ | ↓ | — | ↓ | ↓ | ↓ |
| factor | NF-kB | ↓ | ↓ | ↓ | — | — | — | ↓ | — | ↓ |
| Adhesion | VLA-4 | ↓ | ↓ | ↓ | ↓ | — | ↓ | ↓ | ↓ | ↓* |
| molecules/cell | MMP-9 | | ↑* | ↑* | ↑ | — | ↑ | ↑ | — | ↑* |
| trafficking | ICAM-1 | ↑ | — | — | — | — | ↑ | ↑ | — | — |
| Inflammatory | 1L-1B | ↑ | ↑ | — | ↑ | ↑ | ↑ | ↑ | ↑ | ↑ |
| cytokines/chemokines | CXCR4 | ↓ | ↓* | ↓ | ↓ | ↓* | — | ↓ | ↓* | ↓ |
| and receptors | CCR5 | ↓ | ↓ | ↓ | ↓ | — | ↓ | ↓ | ↓ | ↓* |
| | ApoE | — | — | ↓ | ↓ | — | — | ↓ | ↓ | — |
| | CXCR3 | ↑* | ↑* | ↑ | — | ↑* | ↑ | ↑ | ↑ | — |
| | 1L-8 | ↑ | ↑ | — | — | ↑* | ↑ | ↑ | ↑ | — |
| | CCR3 | — | — | — | — | — | — | ↑ | ↑ | — |
| | TNFa | ↓* | — | ↓ | — | — | — | ↓* | — | ↓ |
| | 1L-12p40 | ↓* | — | ↓ | — | — | — | — | — | — |
| | 1L-12RB2 | ↑* | — | — | — | ↑ | — | — | ↑* | — |
| | 1L-15Ra | — | — | ↓ | — | ↓ | ↓ | ↓* | ↓ | ↓* |

PBMC derived from seven healthy individuals (NS) and two untreated MS patients (MS) were analyzed by gene expression profiling.
Arrows represent specific changes in gene expression (GA treatment/control) by at least two times.
Arrows with asterisks indicate a moderate change in gene expression (>1.5 times and <2 times).

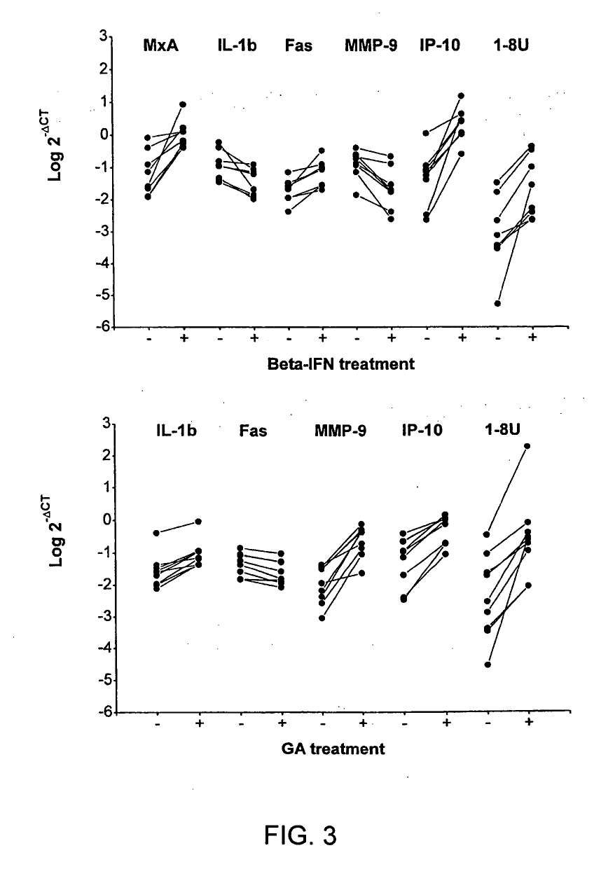

Some variations in responding to beta-IFN or GA among different individuals are noted. To confirm the specific changes in the gene expression induced by beta-IFN or GA, the same sample cDNA preparations were analyzed quantitatively for selected genes (MMP-9, IL-1b, Fas, IP-10, 1-8U and M×A) by real-time PCR using specific primers corresponding to the genes. As shown in FIG. 3, the results were consistent with those by gene expression profiling, confirming characteristic changes in the gene expression measured by the profiling technology.

### Example 9

### Evaluation of Beta-IFN Neutralizing Antibody by Gene Expression Profiling

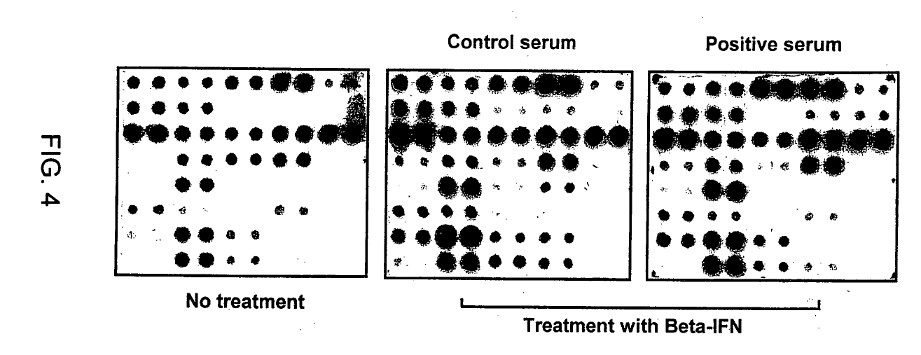

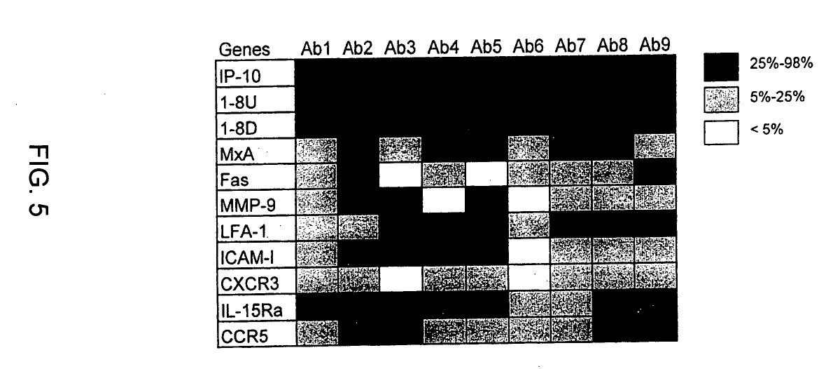

The gene array-based assay was examined for detecting a blocking effect of serum beta-IFN antibody induced by the beta-IFN treatment on the immunomodulatory properties of beta-IFN. Eight MS-derived serum specimens known to contain neutralizing antibodies were tested for a neutralizing effect by reverting the known regulatory effect of beta-IFN on PBMC. The selected serum specimens were obtained from MS patients that had been treated with beta-IFN-1a (Avonex and Rebif) for 2-6 years and were tested positive for beta-IFN binding and neutralizing antibody by ELISA (binding antibody titers 1:256-1:1,024) and by the cytopathic effect reduction (CPE) method (neutralizing antibody titers >1:1,000). Representative PBMC samples that were derived from healthy individuals (NS-5) and represented comprehensive responses to beta-IFN in repeated experiments were selected as indicator cells for all experiments. An untreated MS serum sample that had previously tested negative for beta-IFN antibody served as a control. A representative experiment is shown in FIG. 4. As summarized in FIG. 5, neutralizing antibody selectively reverted the regulatory effect of beta-IFN on 11 of the 17 genes that were susceptible to regulation by beta-IFN, including all interferon-inducible genes, while the remaining six beta-IFN regulated genes (Caspase-3, NF-KB, TNFa, IL-1b, CCR3 and IL-8) were not affected. Serum neutralizing antibody against beta-IFN had no effect on other genes that were not regulated by beta-IFN. The results were reproducible with indicator PBMC derived from different individuals.

### Example 10

Evaluation of Beta-IFN and GA Treatments by Ex Vivo Gene Expression Profiling of PBMC

A group of 18 relapsing-remitting MS patients that had been treated with beta-IFN for 2-7 years and a separate group of 12 relapsing-remitting MS patients that bad received GA treatment for 2-8 years were studied. A group of 15 relapsing-remitting MS patients who had not received any immunomodulatory drugs for at least 24 months prior to the study were included as controls. The clinical characteristics and treatment duration of the patients are shown in Table 3.

TABLE 3
Demographic data and clinical characteristics of
relapsing-remitting patients with MS

| | Study groups | | |
|---|---|---|---|
| | No treatment | Beta-IFN-1a | GA |
| Number of patients | 15 | 18 | 12 |
| Gender (M/F) | 7/8 | 5/13 | 5/7 |
| Age: | | | |
| Mean ± SD | 43.9 ± 8.6 | 41.2 ± 9.3 | 49.6 ± 12.0 |
| Range | 28-64 | 15-52 | 30-68 |
| Disease duration (years): | | | |
| Mean ± SD | 9.7 ± 9.3 | 11.4 ± 6.9 | 14.0 ± 10.5 |
| Range | 3-36 | 4-31 | 2-40 |
| Treatment duration (years): | | | |
| Mean ± SD | N/A | 3.8 ± 1.6 | 3.7 ± 1.9 |
| Range | N/A | 2-7 | 2-8 |
| EDSS: | | | |
| Mean ± SD | 3.6 ± 2.6 | 3.0 ± 2.6 | 3.5 ± 2.9 |
| Range | 0-8 | 0-6.5 | 0-8.5 |

11/18 patients were treated with Avonex® and the remaining 7 patients received Rebif®. Among the beta-IFN treatment group, blood specimens were collected retrospectively from seven patients before treatment (baseline) and after treatment for a direct comparison by ex vivo gene expression profiling. Representative results of self-paired ex vivo analysis are shown in FIG. 6. As summarized in Table 4a and 4b, characteristic changes in the expression of some genes were seen when PBMC specimens obtained 2 years after beta-IFN treatment were analyzed using self-paired baseline PBMC as a reference. It should be noted that some individuals (MS-4 through MS-9) demonstrated a more comprehensive response to the treatment by displaying a high number of affected genes than others (MS-10 through MS-12). The observed differences were not attributable to neutralizing antibody because all nine paired serum specimens were tested negative for beta-IFN antibody.

TABLE 4a
Gene expression profiling in PBMC derived from MS
patients before and after beta-IFN treatment.

| Genes altered | MS4 | MS5 | MS6 | MS7 | MS8 | MS9 | MS10 | MS11 | MS12 |
|---|---|---|---|---|---|---|---|---|---|
| Fas | ↑ | — | ↑* | ↑ | — | ↑* | — | — | — |
| iNos | ↑ | — | ↑ | ↑ | — | — | ↑* | — | — |
| Caspase-3 | — | ↑ | ↑ | — | ↑ | — | — | — | — |
| MMP-9 | — | — | — | ↓ | — | — | — | ↓ | — |
| ICAM-1 | ↑ | — | — | — | ↓ | — | ↑ | — | — |
| LFA-1 | — | — | ↑* | ↑* | — | — | — | — | — |
| 1-8U | ↑ | — | ↑* | ↑ | — | — | — | — | — |
| 1-8D | ↑ | ↑ | ↑* | ↑* | ↑ | — | — | — | — |
| IP-10 | — | — | ↑ | — | ↑ | — | — | — | — |
| IFNγ | ↓ | — | — | ↓ | — | — | — | — | ↓ |
| MxA | ↑* | ↑ | ↑* | ↑* | ↑* | — | — | — | — |
| CXCR3 | — | — | ↑* | ↑* | — | — | — | — | — |
| IL-15 Ra | ↑ | — | ↑* | ↑* | ↑ | ↑* | — | — | — |
| IL-1b | ↓ | ↓* | — | ↓ | ↓ | ↓ | — | — | ↓ |
| IL-8 | ↓ | ↓ | ↓ | ↓ | ↓ | ↓* | — | ↓* | ↓* |
| CXCR4 | ↑ | — | ↑* | ↑ | — | ↑* | — | — | — |
| APOE | ↑ | ↑ | ↑* | — | — | — | — | — | — |
| TNFa | ↓ | — | ↓* | ↓* | — | — | — | ↓* | — |
| CCR3 | ↑ | — | — | ↑ | — | — | — | — | — |
| IL-13 | ↑ | ↑* | — | ↑ | — | ↑* | — | — | — |
| IL-12 RB2 | — | ↑ | ↑* | — | — | — | ↑ | — | — |
| No. of alt. genes | 15 | 8 | 15 | 15 | 8 | 6 | 3 | 3 | 3 |
| Treatment duration | 3 | 5 | 5 | 3 | 5 | 5 | 4 | 3 | 3 |
| (yrs) | | | | | | | | | |

Self-impaired PBMC specimens were obtained in 9 MS patients before and after beta-IFN treatment and analyzed by gene expression profiling. Data are expressed as changes in gene expression (post-treatment/pre-treatment) by greater than 2 times or between 1.5-2 times (asterisks).

TABLE 4b
Gene Expression profiling in PBMC derived from MS
patients before and after GA treatment.

| Genes altered | MS13 | MS14 | MS15 | MS16 | MS17 | MS18 | MS19 |
|---|---|---|---|---|---|---|---|
| Fas | — | — | — | ↑* | — | ↓ | ↑* |
| NV-kB | — | ↓ | — | — | — | ↓ | — |
| iNos | — | — | ↑ | — | ↑* | ↑ | ↑ |
| IP-10 | ↑ | — | — | ↑ | — | — | — |
| 1-8U | — | — | ↑ | ↑ | — | — | — |
| 1-8D | — | ↓ | ↑ | ↑ | — | — | — |
| ICAM-1 | — | — | ↓ | ↓ | ↓ | ↓* | ↓* |
| VLA-4 | — | — | ↓ | ↓ | — | — | — |
| IL-15 Ra | — | ↓ | ↓ | ↓ | ↓ | — | — |
| IL-1b | — | ↓ | ↓ | ↓ | ↓ | ↓ | ↓ |
| IL-8 | — | ↓* | ↓* | ↓ | ↓* | — | ↓ |
| CXCR4 | ↓* | ↓ | — | ↓ | — | — | ↓* |
| APOE | ↓ | ↓* | — | ↓ | ↓* | ↓ | — |
| CCR3 | ↑* | ↑ | — | — | — | — | — |
| IL-13 | — | — | — | ↓ | — | ↓ | — |
| IL-12R B2 | ↑* | ↑* | ↑* | — | ↑* | — | ↑* |
| TGFb1 | — | | — | ↓ | — | — | ↑* |
| TNF | — | ↓ | — | ↓* | — | ↓* | — |
| IL-12 p40 | ↑ | — | ↑ | — | ↑* | ↑ | — |
| No. of genes altered | 6 | 10 | 10 | 14 | 8 | 10 | 8 |
| Treatment duration (yrs) | 2 | 8 | 3 | 3 | 5 | 5 | 2 |

Self-paired PBMC specimens were obtained in 7 MS patients before and after GA treatment and analyzed by gene expression profiling. Data are expressed as changes in gene expression (post-treatment/pre-treatment) by greater than 2 times or between 1.5-2 times (asterisks).

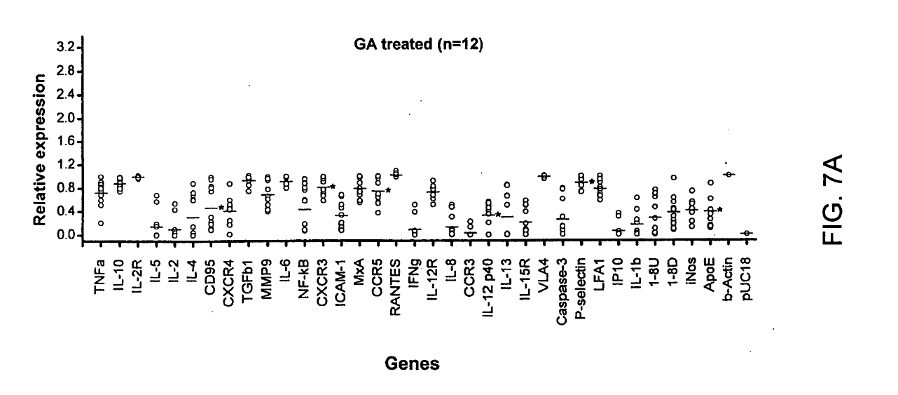

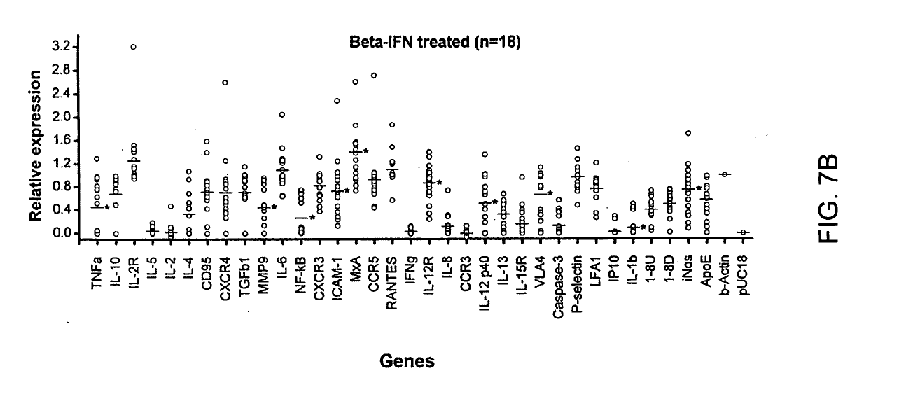

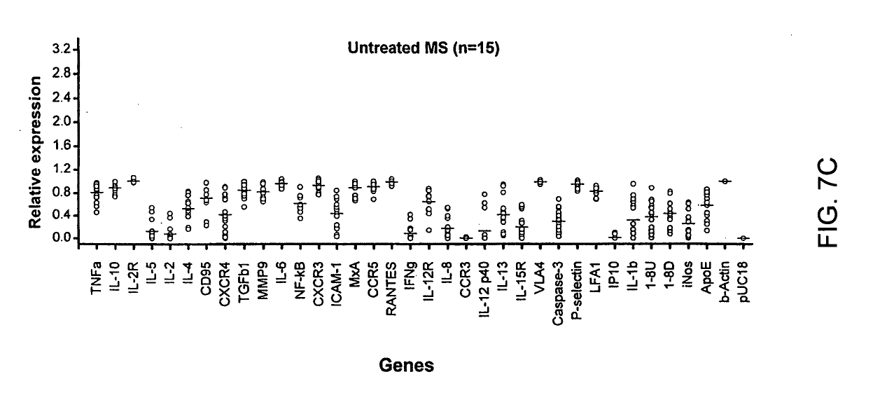

As shown in FIG. 7, ex vivo analysis by gene expression profile between the treatment and control groups revealed significant findings. Comparing to untreated control group, the expression of some genes was significantly altered by the beta-IFN or GA treatment, which was characteristic of each treatment agent. There were also noticeable variations between individuals, which were not, related to neutralizing antibody because only two patients in beta-IFN group had developed measurable beta-IFN antibody. In Table 5, the genes significantly altered by each treatment are listed with p values. The results of group analysis confirmed similar trends in gene expression in self-paired analysis as well as in that induced by in vitro treatments with the exception of CXCFU. There were considerably fewer genes altered by beta-IFN or GA treatment seen in the ex vivo group analysis compared to those affected in vitro by the same drugs (10 vs. 17 for beta-IFN and 6 vs. 19 for GA) and those in self-paired analysis. However, the observed discrepancies were likely due to the fact that changes in expression levels of some of the genes in the group ex vivo analysis did not reach statistical significance and were not listed in Table 5.

Although the number of patient samples analyzed was small, preliminary attempts were made to examine whether some of the genes affected by beta-IFN treatment in MS patients were characteristically associated with clinical benefit. To this end, patients in the beta-IFN treatment group (n=18, Table 5) were broken down into two subgroups according to AEDSS as defined by mean EDSS 2 years prior to treatment and subtracting mean EDSS 2 years after treatment.

TABLE 5
Genes significantly altered by treatment
with Beta-IFN or GA in MS patients

| Treatment group | N | Genes | Change | p value |
|---|---|---|---|---|
| Beta-IFN | 18 | TNFα | ↓ | 0.0052 |
| | | MMP-9 | ↓ | 0.0011 |
| | | NF-κB | ↓ | 0.0004 |
| | | ICAM-1 | ↑ | 0.0251 |
| | | MxA | ↑ | 0.0003 |
| | | IL-12R | ↑ | 0.0245 |
| | | IL-12 p40 | ↑ | 0.0052 |
| | | VLA-4 | ↓ | 0.0045 |
| | | IL-1b | ↓ | 0.0186 |
| | | iNos | ↑ | 0.0018 |
| GA | 12 | Fas | ↓ | 0.0351 |
| | | CXCR3 | ↓ | 0.0151 |
| | | IL-12 p40 | ↑ | 0.0096 |
| | | P-selectin | ↓ | 0.0285 |
| | | ApoE | ↓ | 0.0183 |
| | | CCR5 | ↓ | 0.0268 |

Gene expression level significantly altered by beta-IFN or GA treatment compared to the control group in FIG. 7 are listed here with p values

Although the gene expression profile was similar in both subgroups, the expression of some of the genes affected by beta-IFN treatment, such as IL-1b, IL-12 p40, M×A, exhibited more profound changes in the subgroup with an improved or stable EDSS (mean AEDSS=−0.3, n=10) than that with a worsening EDSS (mean AEDSS=+1.7, n=8). The expression of other genes affected by beta-IFN, such as MMP-9, IL-12R B2 and TNFa, had similar changes in both subgroups.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
U.S. Pat. No. 3,817,837
U.S. Pat. No. 3,850,752
U.S. Pat. No. 3,939,350
U.S. Pat. No. 3,996,345
U.S. Pat. No. 4,277,437
U.S. Pat. No. 4,275,149
U.S. Pat. No. 4,366,241
U.S. Pat. No. 5,578,832
U.S. Pat. No. 5,556,752
U.S. Pat. No. 5,510,270
Adachi, I S., Kumamoto, T., Araki, S., 1990. Elevated soluble interleukin-2 receptor levels in patients with active multiple sclerosis. Ann Neurol28,687-691.
Ahmed, Z., Doward, A. I., Pryce, G., Taylor, D. L., Pocock, J. M., Leonard, J. P., Baker, D., Cuzner, M. L., 2002. A role for caspase-1 and -3 in the pathology of experimental allergic encephalomyelitis: inflammation versus degeneration. Am J Pathol 161, 1577-1586.
Balashov, K. E., Smith, D. R., Khoury, S. J., Hafler, D. A., Weiner, H. L., 1997. Increased interleukin 12 production in progressive multiple sclerosis: induction by activated CD4+ T cells via CD40 ligand. Proc Natl Acad Sci U s1 A 94, 599-603.
Battistini, L., Piccio, L., Rossi, B., Bach, S., Galgani, S., Gdsperini, C., Ottoboni, L., Ciabini, D., Caramia, M. D., Bernardi, G., Laudanna, C., Scarpini, E., McEver, R. P., Butcher, E. C., Borsellino, G., Constantin, G., 2003. CU8+T cells from patients with acute multiple sclerosis display selective increase of adhesiveness in brain venules: a critical role for P-selectin glycoprotein ligand-1. Blood 1101, 4775-4782.
Bonetti, B., Stegagno, C., Cannella, B., Rizzuto, N., MorettQ, G., Raine, C. S., 1999. Activation of NF-kappaB and c-jun transcription factors in multiple sclerosis lesions. Implications for oligodendrocyte pathology. Am J Pathol 155, 1433-1438.
Cannella, B., Raine, C. S., 1995. The adhesion molecule and cytokine profile of multiple sclerosis lesions. Ann Neurol37,424-435.
Comi, G., Filippi, M., Wolinsky, J. S., 2001. EuropeadCanadian multicenter, double-blind, randomized, placebo-controlled study of the effects of glatiramer acetate on magnetic resonance imaging—measured disease activity and burden in patients with relapsing multiple sclerosis. EuropeadCanadian Glatiaamer Acetate Study Group. Ann Neurol49,290-297.
de Jong, B. A., Huizinga, T. W., Bollen, E. L., Uitdehaag, B. M., Bosma, G. P., van Buchem, M. A., Remarque, E. J., Burgmans, A. C., Kalkers, N. F., Polman, C. H., Westendorp, R. G., 2002. Production of IL-1beta and IL-1Ra as risk factors for susceptibility and progression of relapse-onset multiple sclerosis. J Neuroimmunol 126, 172-179.
Duda, P. W., Schrnied, M. C., Cook, S. L., Krieger, J. I., Haflier, D. A., 2000. Glatiramer acetate (Copaxone) induces degenerate, Th2-polarized in immune responses in patients with multiple sclerosis. J Clin Invest 105, 967-976.
Eikelenboom, M. J., Killestein, J., Izeboud, T., Kalkers, N. R., van Lier, R. A., Barkhof, F., Uitdehaag, B. M., Polman, C. H., 2002. Chemokine recqptor expression on T cells is related to new lesion development in multiple sclerosis. J Neuroimmunol 133,225-232.
Elovaara, I., Ukkonen, M., Leppakynnas, M., Lehtimaki, T., Luomala, M., Peltola, J., Dastidar, P., 2000. Adhesion molecules in multiple sclerosis: relation to subtypes of disease and methylprednisolone therapy. Arch Neurol97,546-551.
Fazekas, F., Strasser-Fuchs, S., Kollegger, H., Berger, T., ICristoferitsch, W., Schmidt, H., Enzinger, C., Schiefermeier, M., Schwarz, C., Koqek, B., Reindl, M., Huber, K., Grass, R., Wimmer, G., Vass, K., Pfeiffer, K. H., HartNg, H. P., Schmidt, R., 2001. Apolipoprotein E epsilon 4 is associated with rapid progression of multiple sclerosis. Neurology 57,853-857.
Franciotta, D., Martino, G., Zardini, E., Furlan, R., Bergamaschi, R., Andreoni, L., Cosi, V., 2001. Serum and CSF levels of MCP-1 and IP-10 in multiple sclerosis patients with acute and stable disease and undergoing immunomodulatory therapies. J Neuroimmunol 115, 192-198.
Galboiz, Y., Shapiro, S., Lahat, N., Rawashdeh, H., Miller, A., 2001. Matrix metalloproteinases and their tissue inhibitors as markers of disease subtype and response to interferon-beta therapy in relapsing and secondary-progressive multiple sclerosis patients. Ann Neurol50, 443-451. Gran, B., Tranquill, L. R., Chen, M., Bielekova, B., Zhou, W., Dhib-Jalbut, S., Martin, R., 2000. Mechanisms of immunomodulation by glatiramier acetate. Neurology 55, 1704-1714.
Hogh, P., Oturai, A., Schreiber, K., Blinkenberg, M., Jorgqnsen, O. S., Ryder, L., Paulson, O. B., Sorensen, P. S., Knudsen, G. M., 2000. Apoliprotein E and multiple sclerosis: impact of the epsilon-4 allele on susceptibility, clinic4 type and progression rate. Mult Scler 6,226-230.
Hohnoki, K., Inoue, A., Koh, C. S., 1998. Elevated serum levels of IFN-gamma, IL-4 and TNF-alpha unelevated serum levels of IL-10 in patients with demyelinating diseases during the acute stage. J Neuroimmunol87,27-32.
Hua, L. L., Liu, J. S., Brosnan, C. F., Lee, S. C., 1998. Selective inhibition of human glial inducible nitric oxide synthase by interferon-beta: implications for multiple sclerosis. Ann Neurol43,384-387.
Jacobs, L. D., Cookfair, D. L., Rudick, R. A., Herndon, R. M., Richert, J. R., Salazar, A. M., Fischer, J. S., Goodkin, D. E., Granger, C. V., Simon, J. H., Alam, J. J., Bartoszak, D. M., Bourdette, D. N., Braiman, J., Brownscheidle, C. M., Coats, M. E., Cohan, S. L., Dougherty, D. S., Kinkel, R. P., Mass, M. K., Munschauer, F. E., 3rd, Priore, R. L., Pullicino, P. M., Scherokman, B. J., Whitham, R. H., et al., 1996. Intramuscular interferon beta-1a for disease progression in relapsing multiple sclerosis. The Multiple Sclerosis Collaborative Research Group (MSICRG). Ann Neurol 39,285
Johnson, K. P., Brooks, B. R., Cohen, J. A., Ford, C. C., Golgstein, J., Lisak, R. P., Myers, L. W., Panitch, H. S., Rose, J. W., Schiffer, R. B., 1995. Copolymer 1 reduces relapse rate and improves disability in relapsing-remitting multiple sclerosis: results of a phase III multicenter, double-blind placebo-controlled trial. The Copolymer Multiple Sclerosis Study Group. Neurology 45, 1268-4276.
Killestein, J., Kalkers, N. F., Meilof, J. F., Barkhof, F., van Maier, R. A., Polman, C. H., 2001. TNF-alpha production by CD4(+) T cells predicts long-term increase in lesion load on MRI in MS. Neurology 57, 1129-1131.
Kob, M., Harvey, J., Schautzer, F., Kascha, S., Bibl, D., Eqg, R., Reindl, M., Berger, T., Deiserihammer, F., 2003. A novel and rapid assay for the detection of neutralizing antibodies against interferon-beta. Mult Scler 9,32-35.
Koike, F., Satoh, J., Miyake, S., Yamamoto, T., Kawai, M., Kikuchi, S., Nomura, K., Yokoyama, K., Ota, K., Kanda, T., Fukazawa, T., Yanunura, T., 2003. Microarray analysis identifies interferon beta-regulated genes in multiple sclerosis. J Neuroimmunol 139, 109-118.
Kraus, J., Engelhardt, B., Chatzimanolis, N., Bauer, R., Toffighi, J., Kuehne, B. S., Laske, C., Stolz, E., Frielinghaus, P., Schaefer, C., Blaes, F., Traupe, H., Kaps, M., Oschmann, P., 2002. Cell surface bound and soluble adhesion molecules in CSF and blood in multiple sclerosis: correlation with MRI-measures of subclinical disease severity and activity. J Neuroimmunol 122, 175-185.
Lee, M. A., Palace, J., Stabler, G., Ford, J., Gearing, A., Miller, K., 1999. Serum gelatinase B, TIMP-1 and TIMP-2 levels in multiple sclerosis. A longitudinal clinical and MRI study. Brain 122 (Pt 2), 191-197.
Leppert, D., Waubant, E., Burk, M. R., Oksenberg, J. R., Hauser, S. L., 1996. Interferon beta-1 b inhibits gelatinase secretion and in vitro migration of human T cells: a possible mechanism for treatment efficacy in multiple sclerosis. Ann Neurol40, 846-852.
Lock, C., Hermans, G., Pedotti, R., Brendolan, A., Schadt, E., Garren, H., Langer-Gould, A., Strober, S., Cannella, B., Allard, J., Klonowski, P., Austin, A., Lad, N., Kaminski, N., Galii, S. J., Oksenberg, J. R., Raine, C. S., Heller, R., Steinman, L., 2002. Gene-microarray analysis of multiple sclerosis lesions yields new targets validated in autoimmune encephalomyelitis. Nat Med d, 500-508.
Martinez-Caceres, E. M., Espejo, C., Brieva, L., Pericot, I., Tintore, M., Saez-Torres, I., Montalban, X., 2002. Expression of chemokine receptors in the different clinical forms of multiple sclerosis. Mult Scler 8,390-395.
Misu, T., Onodera, H., Fujihara, K., Matsushima, K., Yoshie, O., Okita, N., Takase, S., Itoyama, Y., 2001. Chemokine receptor expression on T cells in blood and cerebrospinal fluid at relapse and remission of multiple sclerosis: imbalance of Th1-Th2-associated chemokine signaling. J Neuroimmunol 114,207-212.
Muraro, P. A., Leist, T., Bielekova, B., McFarland, H. F., 2Q00. VLA-41CD49d down-regulated on primed T lymphocytes during interferon-beta therapy in multiple sclerosis. J Neuroimmunol 1 11, 186-194.
Navikas, V., Matusevicius, D., Soderstrom, M., Fredrikson, S., Kivisakk, P., Ljungdahl, A., Hojeberg, B., Link, H., 1996. Increased interleukin-6 mRNA expression in blood and cerebrospinal fluid mononuclear cells in multiple sclerosis. J Neuroimmuno 64, 63-69.
Neuhaus, O., Farina, C., Yassouridis, A., Wiendl, H., Then Bergh, F., Dose, T., Wekerle, H., Hohlfeld, R., 2000. Multiple sclerosis: comparison of copolymer-1-reactive T cell lines from treated and untreated subjects reveals cytokine shift from T helper 1 to T helper 2 cells. Proc Natl Acad Sci USA 97,7452-7457. PRISMS (Prevention of Relapses and Disability by InterferQn beta-I a Subcutaneously in Multiple Sclerosis) Study Group. 1998. Randomised double-blind placebo-controlled study of interferon beta-1a in relapsing remitting multiple sclerosis. Lancet 352, 1498-1504.
Rieckmann, P., Albrecht, M., Kitze, B., Weber, T., Tumani, H., Broocks, A., Luer, W., Helwig, A., Poser, S., 1995. Tumor necrosis factor-alpha messenger RNA expression in patients with relapsing-remitting multiple sclerosis is associated with disease activity. Ann Neurol37, 82-88.
Rudick, R. A., Ransohoff, R. M., Peppler, R., VanderBrug Mbdendorp, S., Lehmann, P., Alam, J., 1996. Interferon beta induces interleukin-10 expression: relevance to multiple sclerosis. Ann Neurol40, 618-627.
Schonrock, L. M., Gawlowski, G., Bruck, W., 2000. Interleukin-6 expression in human multiple sclerosis lesions. Neurosci Lett 294,45-48.
Schrijver, H. M., van As, J., Crusius, J. B., Dijkstra, C. D., Uitdehaag, B. M., 2003. Interleukin (IL)-1 gene polymorphisms: relevance of disease severity associated alleles with IL-1 beta and IL-1 ra production in multiple sclerosis. Mediators Inflamm 12,89-94.
Sharief, M. K., Hentges, R., 1991. Association between tumpr necrosis factor-alpha and disease progression in patients with multiple sclerosis. N Engl J Med 325,467-472.
Simpson, J., Rezaie, P., Newcombe, J., Cumer, M. L., Malq, D., Woodroofe, M. N., 2000. Expression of the beta-chemokine receptors CCR2, CaR3 and CCRS in multiple sclerosis central nervous system tissue. J Neuroimmunol 108, 192-200.
Sivieri, S., Ferrarini, A. M., Gallo, P., 1998. Multiple sclerosis: IL-2 and sIL-2R levels in cerebrospinal fluid and serum. Review of literature and critical analysis of ELISA pitfalls. Mult Scler 4,7-11.
Sorensen, T. L., Tani, M., Jensen, J., Pierce, V., Lucchinetti, C., Folcik, V. A., Qin, S., Rottman, J., Sellebjerg, F., Strieter, R. M., Frederiksen, I J. L., Ransohoff, R. M., 1999. Expression of specific chemokines and chemokine receptors in the central nervous system of multiple sclerosis patients. J Clin Invest 103, 807-815.
Strunk, T., Bubel, S., Mascher, B., Schlenke, P., Kirchner, B., Wandinger, K. P., 2000. Increased numbers of CCRS+interferon-gamma- and tqunor necrosis factor-alpha-secreting T lymphocytes in multiple sclerosis patients. Ann Neurol47,269-273.
Sturzebecher, S., Wandinger, K. P., Rosenwald, A., Sathyanboorthy, M., TZOU, A., Mattar, P., Frank, J. A., Staudt, L., Martin, R., McFarland, H. F., 2003. Expression profiling identifies responder and non-responder phenotypes to interferon-beta in multiple sclerosis. Brain 126, 1419-1429. The IFNB Multiple Sclerosis Study Group. 1993. Interferon beta-1 b is effective in relapsing-remitting multiple sclerosis. I. Clinical results of a multicenter, randomized, double-blind, placebo-controlled trial. Neurology 43,655-661. The IFNB Multiple Sclerosis Study Group. 1996. Neutralizing antibodies during treatment of multiple sclerosis with interferon beta-1 b: experience during the first three years. Neurology 47,889-894.
Vallittu, A. M., Halminen, M., Peltoniemi, J., ilonen, J., Jukunen, I., Salmi, A., Eralinna, J. P., 2002. Neutralizing antibodies reduce MxA protein induction in interferon-beta-1 a-treated MS patients. Neurology 58, 1786-1790.
van Boxel-Dezaire, A. H., Hoff, S. C., van Oosten, B. W., VGrweij, C. L., Drager, A. M., Ader, H. J., van Houwelingen, J. C., Barkhof, F., Polma, C. H., Nagelkerken, L., 1999. Decreased interleukin-10 and increased interleukin-12˜40 mRNA are associated with disease activity and characterize different disease stages in multiple sclerosis. Ann Neurol45,695-703.
van Boxel-Dezaire, A. H., Smits, M., van Trigt-Hoff, S. C., qillestein, J., van Houwelingen, J. C., Polman, C. H., Nagelkerken, L., 2001. Cytokine and IL-12 receptor mRNA discriminate between different clinical subtypes in multiple sclerosis. J Neuroimmunol 120, 152-160.
Wandinger, K. P., Sturzebecher, C. S., Bielekova, B., Detore, G., Rosenwald, A., Staudt, L. M., McFarland, H. F., Martin, R., 2001. Complex immunomodulatory effects of 29 interferon-beta in multiple sclerosis include the upregulation of T helper 1-associated marker genes. Ann Neurol50, 349-357.
Weinstock-Guttman, B., Badgett, D., Patrick, K., Hartrich, L., Santos, R., Hall, D., Baier, M., Feichter, J., Ramanathan, M., 2003. Genomic effects of IFN-beta in multiple sclerosis patients. J Immunol 171,2694-2702.
Wiesemann, E., Klatt, J., Sonmez, D., Blasczyk, R., Heidqnreich, F., Windhagen, A., 2001. Glatiramer acetate (GA) induces IL-13/IL-5 secretion in naive T cells. J Neuroimmunol 119, 137-144.
Yong, V. W., Chabot, S., Stuve, O., Williams, G., 1998. Interferon beta in the treatment of multiple sclerosis: mechanisms of action. Neurology 91,682-689.
Zang, Y. C., Halder, J. B., Samana A. K., Hong, J., Rivera, V. M., Zhang, J. Z., 2001. Regulation of chemokine receptor CCR5 and production of RANTES and MIP-1 alpha by interferon-beta. J Neuroimmunol 112, 174-180.
Zang, Y. C., Samanta, A. K., Halder, J. B., Hong, J., Tejada-Fimon, M. V., Rivera, V. M., Zhang, J. Z., 2000a. Aberrant T cell migration toward WNTES and MIP-1 alpha in patients with multiple sclerosis. Overexpression of chqmokine receptor CCRS. Brain 123 (P t 9), 1874-1882.
Zang, Y. C., Yang, D., Hong, J., Tejada-Simon, M. V., Rivefa, V. M., Zhang, J. Z., 2000b. Immunoregulation and blocking antibodies induced by interferon beta treatment in MS. Neurology 55,397-404.
Zhang, J., Hutton, G., Zang, Y., 2002. A comparison of the mechanisms of action of interferon beta and glatiramer acetate in the treatment qf multiple sclerosis. Clin Ther 24, 1998-2021.
Zipp, F., Otzelberger, K., Dichgans, J., Martin, R., Weller, M., 1998a. Serum CD95 of relapsing remitting multiple sclerosis patients protects from CD95-mediated apoptosis. J Neuroimmunol86, 151-154.
Zipp, F., Weller, M., Calabresi, P. A., Frank, J. A., Bash, C. N., Dichgans, J., McFarland, H. F., Martin, R., 1998b. Increased serum levels of soluble CD95 (APO-1/Fas) in relapsing-remitting multiple sclerosis. Ann Neurol43, 116-120.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method of monitoring a multiple sclerosis patient taking beta-IFN comprising the steps of:
obtaining a sample of peripheral blood mononuclear cells from the patient;
isolating RNA from the sample; and
determining the relative expression profile in the isolated RNA of at least four individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and
comparing the relative expression profile of the individual nucleic acids to a control sample, wherein the beta-IFN is predicted to be therapeutically effective if the relative expression profile is characteristic of a beta-IFN therapy response.

2. The method of claim 1, wherein determining the relative expression of individual nucleic acids in the RNA comprises the steps of:
providing a plurality of probes bound to a solid surface, at least four of said plurality of probes being complementary to sequences selected from the group of nucleic acids consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34;
contacting the probes with the RNA obtained from the sample of peripheral blood mononuclear cells; and
detecting binding of the RNA to the probes; thereby identifying differences in relative expression of the nucleic acids.

3. The method of claim 2, wherein the detecting of binding comprises detecting fluorescent or radioactive labels.

4. The method of claim 2, wherein the solid surface is glass or nitrocellulose.

5. The method of claim 1, wherein at least one of the individual nucleic acids is selected from the group consisting of SEQ ID NO: 15, SEQ ID NO:29, SEQ ID NO:31, and SEQ ID NO:32;
and at least one of the individual nucleic acids is selected from the group consisting of SEQ ID NO:1, SEQ ID NO:2, SEQ ID NO:5, SEQ ID NO:6, SEQ ID NO:18, SEQ ID NO:19, SEQ ID NO:22, SEQ ID NO:23, and SEQ ID NO:30.

6. The method of claim 1, wherein the at least four individual nucleic acids are SEQ ID NO:2, SEQ ID NO:15, SEQ ID NO:18, and SEQ ID NO:22.

7. The method of claim 1, wherein a relative change in expression as compared to the control sample of at least one nucleic acid selected from the group consisting of SEQ ID NO:1, SEQ ID NO:10, SEQ ID NO:12, SEQ ID NO:14, SEQ ID NO:15, SEQ ID NO:19, SEQ ID NO:22, SEQ ID NO:25, SEQ ID NO:30, and SEQ ID NO:33 is characteristic of the beta-IFN therapy response.

8. The method of claim 1, wherein relative decreased expression as compared to the control sample of at least one nucleic acid selected from the group consisting of SEQ ID NO:1, SEQ ID NO:10, SEQ ID NO:12, SEQ ID NO:25, and SEQ ID NO:30 is characteristic of the beta-IFN therapy response.

9. The method of claim 8, wherein the relative decrease is at least about 1.5-fold.

10. The method of claim 1, wherein relative increased expression as compared to the control sample of at least one nucleic acid selected from the group consisting of SEQ ID NO:14, SEQ ID NO:15, SEQ ID NO:19, SEQ ID NO:22, and SEQ ID NO:33 is characteristic of the beta-IFN therapy response.

11. The method of claim 10, wherein the relative increase is at least about 1.5-fold.

12. A method of predicting treatment response of a multiple sclerosis patient to beta-IFN therapy comprising the steps of:
obtaining a sample of peripheral blood mononuclear cells from the patient;
contacting the ample of peripheral blood mononuclear cells with a therapeutically effective amount of beta-IFN;
isolating RNA from the sample;
determining the relative expression profile in the isolated RNA of at least four individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and
comparing the relative expression profile of the individual nucleic acids to a control sample, wherein the beta-IFN is predicted to be therapeutically effective if the relative expression profile is characteristic of a beta-IFN therapy response.

13. A method of screening a multiple sclerosis patient for the presence of neutralizing antibody to beta-IFN comprising the steps of:
obtaining a sample of peripheral blood mononuclear cells from the patient;
isolating RNA from the sample;
determining the relative expression profile in the isolated RNA of at least four individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:1, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and
comparing the relative expression profile of the individual nucleic acids to a control sample, wherein neutralizing antibody to beta-IFN is present if the relative expression profile is characteristic of a blocked beta-IFN therapy response.

14. A method of monitoring a multiple sclerosis patient taking beta-IFN comprising the steps of:
obtaining a sample of peripheral blood mononuclear cells from the patient;
isolating RNA from the sample; and
determining the relative expression profile in the isolated RNA of at least two individual nucleic acids, wherein at least one individual nucleic acid is selected from the group consisting of SEQ ID NO:15, SEQ ID NO:29, SEQ ID NO:31, and SEQ ID NO:32, and at least one individual nucleic acid is selected from the group consisting of SEQ ID NO:1, SEQ ID NO:2, SEQ ID NO:5, SEQ ID NO:6, SEQ ID NO:18, SEQ ID NO:19, SEQ ID NO:22, SEQ ID NO:23, and SEQ ID NO:30; and
comparing the relative expression profile of the individual nucleic acids to a control sample, wherein the beta-IFN is predicted to be therapeutically effective if the relative expression profile is characteristic of a beta-IFN therapy response.

15. A method of monitoring a multiple sclerosis patient taking glatiramer acetate comprising the steps of:
obtaining a sample of peripheral blood mononuclear cells from the patient;
isolating RNA from the sample;
determining the relative expression profile in the isolated RNA of at least three individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and
comparing the relative expression profile of the individual nucleic acids to a control sample, wherein the glatiramer acetate is therapeutically effective if the relative expression profile is characteristic of a glatiramer acetate therapy response.

16. The method of claim 15, wherein determining the relative expression of individual nucleic acids in the RNA comprises the steps of:
providing a plurality of probes bound to a solid surface, at least three of said plurality of probes being complementary to sequences selected from the group of nucleic acids consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34;
contacting the probes with the RNA obtained from the sample of peripheral blood mononuclear cells; and
detecting binding of the RNA to the probes; thereby identifying differences in relative expression of the nucleic acids.

17. The method of claim 15, wherein a relative change in expression as compared to a control sample of at least one nucleic acid selected from the group consisting of SEQ ID NO:7, SEQ ID NO:13, SEQ ID NO:16, SEQ ID NO:22, SEQ ID NO:27, and SEQ ID NO:34 is characteristic of the glatiramer acetate therapy response.

18. The method of claim 15, wherein relative decreased expression as compared to a control sample of at least one nucleic acid selected from the group consisting of SEQ ID NO:7, SEQ ID NO:13, SEQ ID NO:16, SEQ ID NO:27, and SEQ ID NO:34 is characteristic of the glatiramer acetate therapy response.

19. The method of claim 18, wherein the relative decrease is at least about 1.5-fold.

20. The method of claim 15, wherein relative increased expression as compared to a control sample of SEQ ID NO:22 is characteristic of the glatiramer acetate therapy response.

21. The method of claim 18, wherein the relative increase is at least about 1.5-fold.

22. The method of claim 13, wherein the at least three individual nucleic acids are SEQ ID NO:1, SEQ ID NO:2, and SEQ ID NO: 18.

23. A method of predicting treatment response of a multiple sclerosis patient to glatiramer acetate therapy comprising the steps of:
obtaining a sample of peripheral blood mononuclear cells from the patient;
contacting the sample of peripheral blood mononuclear cells with a therapeutically effective amount of glatiramer acetate;
isolating RNA from the sample;
determining the relative expression profile in the isolated RNA of at least three individual nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34; and
comparing the relative expression profile of the individual nucleic acids to a control sample, wherein the glatiramer acetate is predicted to be therapeutically effective if the relative expression profile is characteristic of a glatiramer acetate therapy response.

24. An array comprising nucleic acid probes attached to a solid surface, wherein the nucleic acid probes are complementary to at least five of the nucleic acids selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 SEQ ID NO:11, SEQ ID NO:12, SEQ ID NO:13, SEQ ID NO:14, SEQ ID NO:15, SEQ ID NO:16, SEQ ID NO:17; SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 SEQ ID NO:28, SEQ ID NO:29, SEQ ID NO:30, SEQ ID NO:31, SEQ ID NO:32, SEQ ID NO:33, and SEQ ID NO:34.

25. The array of claim 24, wherein the solid surface is nitrocellulose or glass.
